(19) United States
(12) Patent Application Publication
(10) Pub. No.: US 2002/0119581 A1
(43) Pub. Date: Aug. 29, 2002

(54) Detection of analytes

(76) Inventors: George Y. Daniloff, Mountain View, CA (US); Aristotle G. Kalivretenos, Columbia, MD (US); Alexandre V. Nikolaitchik, Damascus, MD (US); Edwin F. Ullman, Atherton, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/028,331

(22) Filed: Dec. 28, 2001

Related U.S. Application Data
(63) Continuation-in-part of application No. 09/754,219, filed on Jan. 5, 2001.

(51) Int. Cl. G01N 33/543

(52) U.S. Cl. 436/518

(57) ABSTRACT

Disclosed are methods for detecting analytes with indicator systems which may undergo a molecular configurational change upon exposure to the analyte. The configurational change affects a detectable quality associated with the indicator system, thereby allowing detection of the presence or concentration of the analyte.

### CROSS-REFERENCE TO RELATED APPLICATIONS

This application is a continuation-in-part of application Ser. No. 09/754,219 filed Jan. 5, 2001.

### STATEMENT REGARDING FEDERALLY SPONSORED RESEARCH OR DEVELOPMENT

Not applicable.

### BACKGROUND OF THE INVENTION

1. Field of the Invention

The present invention relates to the detection of the presence or concentration of an analyte. More particularly, the invention relates to detecting analytes with indicator systems which may undergo a molecular configurational change upon exposure to the analyte. The configurational change affects a detectable quality associated with the indicator system, thereby allowing detection of the presence or concentration of the analyte.

2. Description of the Related Art

U.S. Pat. No. 5,503,770 (James, et al.) is directed to a fluorescent boronic acid-containing compound that emits fluorescence of a high intensity upon binding to saccharides, including glucose. The fluorescent compound has a molecular structure comprising a fluorophore, at least one phenylboronic acid moiety and at least one amine-providing nitrogen atom where the nitrogen atom is disposed in the vicinity of the phenylboronic acid moiety so as to interact intramolecularly with the boronic acid. Such interaction thereby causes the compound to emit fluorescence upon saccharide binding. U.S. Pat. No. 5,503,770 describes the compound as suitable for detecting saccharides. See also T. James, et al., J. Am. Chem. Soc. 117(35):8982-87 (1995).

Nature Biotechnology 16, 49-53 (1998) is directed to allele discrimination utilizing molecular beacons, i.e., hairpin-shaped oligonucleotide probes labeled with a fluorophore/quencher pair. Upon binding to the target, the probe undergoes a configurational reorganization that restores the fluorescence of the internally quenched fluorophore. However, because the strength of DNA base-pairing is relatively high at ambient temperature, and the molecular beacon probe in use must undergo a large configurational change (through essentially 180°), that system cannot readily be used to continuously detect fluctuating analyte concentrations in real time.

There remains a need in the art for indicator systems which are capable of detecting the presence or concentration of an analyte with greater sensitivity, and which may also use a wide variety of detection systems, and which may also be used for the real time detection of analytes whose concentration may be fluctuating.

### BRIEF SUMMARY OF THE INVENTION

In one aspect, the present invention is directed to a method for detecting the presence or concentration of a polyhydroxyl analyte in a sample, which comprises:

a) exposing the sample to an indicator system having

i) a first recognition element capable of forming a covalent bond in a reversible fashion with said analyte, and either A) a second recognition element capable of forming a covalent bond in a reversible fashion to said analyte bound to the first recognition element, or B) a ligand element capable of interacting in a reversible fashion with the first recognition element in the absence of said analyte, said ligand element optionally further comprising a label that produces a detectable quality that is modulated by the interaction of the ligand element with the recognition element, wherein the portion of the indicator system containing said first recognition element is covalently or non-covalently linked to the portion of the indicator system containing said second recognition element or said ligand element; and

ii) a detection system which comprises at least one of A) a donor/acceptor system which produces a detectable quality that changes in a concentration-dependent manner when said indicator system is exposed to said analyte, or B) said labeled ligand element; and

b) measuring any change in said detectable quality to thereby determine the presence or concentration of said analyte in said sample.

In another aspect, the present invention is directed to indicator systems for carrying out the methods set forth above.

### BRIEF DESCRIPTION OF THE DRAWINGS

FIG. 1 shows the normalized fluorescence emission (I/Io @ 535 nm) of the compounds described in Example 1.

FIG. 2 shows the normalized fluorescence emission (I/Io @ 535 nm) of the compounds described in Example 2.

FIG. 3 shows the fluorescence emission (I at 518 nm) of the indicator system described in Example 3.

FIG. 4 shows the fluorescence emission (I at 545 nm) of the indicator system described in Example 4.

FIG. 5 shows the fluorescence emission (I at 532 nm) of the indicator system described in Example 5.

FIG. 6 shows the fluorescence emission (I at 450 nm) of the indicator system described in Example 6.

FIG. 7 shows the normalized fluorescence emission (I at 430 nm) of the indicator system described in Example 6.

FIG. 8 shows the absorbance spectra of the indicator system described in Example 7.

FIG. 9 shows the ratio of absorbance (A (565 nm)/A (430 nm)) of the indicator system described in Example 7.

FIG. 10 shows the normalized fluorescence (I/Iₒ) at 550 nm of the indicator system described in Example 7.

### DETAILED DESCRIPTION OF THE INVENTION

In one aspect, the present invention provides a way to detect the presence or concentration of an analyte using an indicator system which may undergo a configurational change upon interaction with the analyte. The indicator system has a detectable quality that changes when the indicator system undergoes the configurational change, which is indicative of the presence or concentration of the analyte.

Many analytes may be detected according to the present invention. Suitable analytes include molecular analytes (which may be defined as a molecule consisting of covalent bonds, as opposed to, e.g., a metal ion or metal complex comprised of coordinative bonds); carbohydrates; polyhydroxyl compounds, especially those having vicinal hydroxy groups, such as free sugars (e.g., glucose, fructose, lactose, etc.) and sugars bound to lipids, proteins, etc.; small molecule drugs; hormones; oxygen; carbon dioxide; various ions, such as zinc, potassium, hydrogen, carbonate, etc. The present invention is especially suited to detection of small analytes, particularly less than 5000 Daltons.

In one embodiment, the present invention may be carried out using an indicator system which has at least two recognition elements for the analyte to be detected, which are oriented such that upon interacting with the analyte capable of two-site interaction, the indicator system undergoes the configurational change. The indicator system also has a detection system associated therewith, which has a detectable quality which changes when the indicator system interacts with the analyte. Upon interaction with the analyte, the recognition elements may assume a configuration where they are either closer together or farther apart, or restricted in their freedom of molecular motion which in turn may affect the signal, than their configuration in the absence of the analyte. That change in configuration may cause the change in the detectable quality.

In another embodiment, the present invention may be carried out using an indicator system which has at least one recognition element for the analyte to be detected, as well as a ligand element. The ligand element is capable of reversible interaction with the recognition element, and competes with the analyte for interaction with the recognition element. When the recognition element and the ligand element interact in the absence of the analyte, the detection system will have a different preferred configuration or relative orientation than when the analyte interacts with the recognition element, causing displacement of the ligand element from the recognition element. That change in configuration causes the change in the detectable quality. In certain embodiments, the ligand element may also be part of the detection system. For example, the ligand element may also be a quencher, whose effect is removed when the analyte interacts with the recognition element. Further, the ligand element may comprise, for example, a detectable label whose characteristics (e.g., spectral profile) differs depending upon whether or not the ligand element interacts with the recognition element.

With respect to either embodiment described above, suitable recognition elements include moieties which are capable of a preferably reversible interaction with the analyte to be detected. It will be understood that the term “interaction” can include a wide variety of physical and chemical interactions, such as charge interactions, hydrogen bonding, covalent bonding, etc. It is especially preferred that the interaction between the recognition element(s) and analyte, and between the ligand element (if present) and the recognition element, be the formation of one or more covalent bonds in a reversible fashion. In this context, a covalent bond preferably means a bond between two atoms where one electron is provided by each atom, and excludes hydrogen bonding, ionic bonding, and coordinative or dative bonding involving donation of two electrons from one of the two atoms. It is preferred that the interaction be relatively weak, e.g., having a dissociation constant of above about 10^{31 6} M. Several suitable recognition elements are known, and preferably include boronic acid, boronate ion, arsenious acid, arsenite ion, telluric acid, tellurate ion, germanic acid, germanate ion, etc., all of which are known to recognize vicinal diols such as glucose and other carbohydrates. When the analyte is glucose, boronic acid is the most preferred recognition element.

In the embodiment where the indicator system includes a ligand element, such element should be capable of interaction with the recognition element and designed depending on the dynamic range of the target analyte. Choice of the ligand element will depend upon the analyte and the recognition element, within the guidelines mentioned above. In a preferred embodiment, when the analyte is a vicinal diol such as glucose and the recognition element is a boronic acid, the ligand element is preferably a moiety capable of forming a bond with the recognition element (such as an ester bond) in a reversible fashion. Such ligand elements include an aromatic diol (e.g., a catechol), a lactate, an alpha-hydroxy acid, tartaric acid, malic acid, diethanolamine, a β-aminoalcohol, glucose, a polyhydroxy compound, and a vicinal hydroxy-containing compound, all optionally substituted. In another embodiment, the ligand element may also be part of the detection system. For example, the ligand element may also be capable of modulating the fluorescence of a fluorophore associated with the indicator system. When the ligand element interacts with the recognition element, it is in a configuration where it may, e.g., effectively quench the fluorophore. When the ligand element is displaced from the recognition element by the analyte, the ligand is no longer in a configuration to quench the fluorophore (see Example 6). The reverse case could also be true in another embodiment (the quencher unable to interact with the fluorophore when interacting with the recognition element).

In use, the present indicator systems preferably exist in dynamic equilibrium between the configurational states described herein. More preferably, there is a relatively weak binding and a high rate of interaction, allowing faster equilibration in the presence of free analyte. Consequently, use of the present invention preferably permits real-time analyte detection over a wide range of conditions, especially detection of an analyte whose concentration is fluctuating. The present invention generally will not require the use of substantial temperature changes in carrying out the methods described herein. That is, the present methods may be performed at substantially ambient temperature, which means the temperature at which the analyte sample is found under normal conditions. It will be understood that ambient temperature will vary widely depending on the analyte and its environment. For example, ambient temperature may include room temperature or colder; up to about 45° C. for many in vivo applications; and up to about 80° C. or higher for, e.g., certain fermentation applications.

The indicator systems of the present invention include a detection system which has a detectable quality that changes in a concentration-dependent manner when the indicator system is exposed to an analyte. The detection system preferably comprises a donor/acceptor system, which means a pair of different groups that interact to provide a signal, wherein a change in the distance between the groups changes a characteristic of the signal. Preferably, the signal is an electromagnetic or electrochemical signal (e.g., a charge transfer pair which provides a different electrochemical potential when in close proximity).

Many such qualities/systems are known and may be used in the present invention. For example, the indicator system may include a luminescent (fluorescent or phosphorescent) or chemiluminescent label, an absorbance based label, etc., which undergoes a change in the detectable quality when the indicator system undergoes the configurational change. The detection system may comprise a donor moiety and an acceptor moiety, each spaced such that there is a detectable change when the indicator system interacts with the analyte.

The detectable quality may be a detectable spectral change, such as changes in fluorescent decay time (determined by time domain or frequency domain measurement), fluorescent intensity, fluorescent anisotropy or polarization; a spectral shift of the emission spectrum; a change in time-resolved anisotropy decay (determined by time domain or frequency domain measurement), a change in the absorbance spectrum, etc.

The detection system may comprise a fluorophore and a moiety that is capable of quenching the fluoresence of the fluorophore. In that embodiment, the indicator system may be constructed in two ways. First, it may be constructed such that in the absence of analyte, the fluorophore and quencher are positioned sufficiently close to each other such that fluorescent emission is effectively quenched. Upon interaction with the analyte, the configuration of the indicator system changes, resulting in the separation of the fluorophore/quencher pair sufficient to allow dequenching of the fluorophore. Alternatively, the indicator system may be constructed such that in the absence of analyte, the fluorophore and quencher are positioned sufficiently distant from each other such that the fluorophore is capable of emitting fluorescence. Upon interaction with the analyte, the configuration of the indicator system changes, and the fluorophore/quencher pair is brought sufficiently close to allow quenching of the fluorophore. As used herein, the fluorophore/quencher pair is intended to include the situation where both members of the pair are fluorophores, either the same or different, but when the indicator system is in the quenching configuration, one fluorophore affects the fluorescence of the other, as by proximity effects, energy transfer, etc.

Many fluorophore/quencher pairs are known and are contemplated by the present invention. For example, it is known that DABCYL will efficiently quench many fluorophores, such as coumarin, EDANS, fluorescein, Lucifer yellow, BODIPY™ Eosine, tetramethylrhodamine, Texas Red™, etc.

It will be understood that the fluorescence emitted from the fluorophore may be quenched through a variety of mechanisms. One way is by quenching via photoinduced electron transfer between the fluorophore and quencher (see Acc. Chem. Res. 1994, 27, 302-308, incorporated by reference). Quenching may also occur via an intersystem crossing caused by a heavy atom effect or due to the interaction with a paramagnetic metal ion, in which case the quencher may contain a heavy atom such as iodine, or a paramagnetic metal ion such as Cu⁺² (see, e.g., J.Am.Chem.Soc. 1985, 107, 7783-7784, and J.Chem.Soc. Faraday Trans., 1992, 88, 2129-2137, both incorporated by reference). The quenching may also take place via a ground state complex formation between the fluorophore and quencher, as described in Nature Biotechnology, 1998, 16, 49-53, incorporated by reference. Another quenching mechanism involves fluorescence resonance energy transfer (FRET) as described in, e.g., Meas. Sci. Technol. 10 (1999) 127-136 and JACS 2000, 122, 10466-10467, incorporated by reference.

Another class of moieties useful in the present detection system includes those whose absorbance spectrum changes upon the change in molecular configuration, including Alizarin Red-S, etc.

Suitable indicator systems for use in the present invention include compositions of matter which contain one of the following schematic structures:

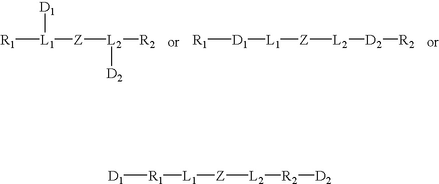

wherein:

-R₁ is one or more recognition elements for said analyte;

-R₂ is either i) one or more recognition elements for said analyte, or ii) an optionally labeled ligand element;

-D₁ and D₂ together comprise a detection system which comprises an energy donor/acceptor system, has a detectable quality that changes in a concentration-dependent manner when said indicator molecule interacts with the analyte, or D₁ and D₂ may be absent when R₂ is a labeled ligand element;

-L₁ and L₂ are the same or different and comprise linking groups of sufficient length and structure to allow the interactions and detectable quality changes to take place; and

Z is a covalent or non-covalent linkage between L₁ and L₂.

The recognition elements, ligand element, and detection system have already been described. The linking groups L₁ and L₂ have a length and structure sufficient to allow the stated interactions and changes to occur. It will be recognized that the exact nature of the linking groups will depend upon the structures of the other elements of the indicator system. Linkers can be designed for structural rigidity, molecular distance, charge interaction, etc., which can be used to optimize the reversible analyte detection system interaction, as shown in the examples.

The Z component of the present indicator systems represents a preferably covalent linkage between L₁ and L₂. The indicator system may have the form of a single molecule or macromolecule.

L₁ and L₂ may take a wide variety of forms. For example, suitable linking groups include alkyl, aryl, polyamide, polyether, polyamino, polyesters and combinations thereof, all optionally substituted.

The indicator systems of the present invention, if soluble, may be used directly in solution if so desired. On the other hand, if the desired application so requires, the indicator systems may be immobilized (such as by mechanical entrapment or covalent or ionic attachment) onto or within an insoluble surface or matrix such as glass, plastic, polymeric materials, etc. When the indicator system is entrapped within, for example, a polymer, the entrapping material preferably should be sufficiently permeable to the analyte to allow suitable interaction between the analyte and the indicator system.

If the indicator system is sparingly soluble or insoluble in water, yet detection in an aqueous medium is desired, the indicator system may be co-polymerized with a hydrophilic monomer to form a hydrophilic macromolecule as described in co-pending U.S. application Ser. No. 09/632,624, filed Aug. 4, 2000, the contents of which are incorporated herein by reference.

It will be understood that the present indicator systems may take many forms chemically. For example, the entire indicator system may be one molecule, of relatively small size. Or, the individual components of the indicator system could be part of a macromolecule. In the latter instance, components of the system could be incorporated into the same polymer, or could be associated with separate cross-linked polymers. For example, separate monomers containing a fluorophore/ligand element adduct and a quencher/recognition element adduct can be copolymerized to form an indicator system polymer (see Example 5). Alternatively, the monomers may be polymerized separately to form separate polymer chains, which may then be cross-linked to form the indicator system.

Many uses exist for the indicator systems of the present invention, including uses as indicators in the fields of energy, medicine and agriculture. For example, the indicator systems can be used as indicator molecules for detecting sub-levels or supra-levels of glucose in blood or urine, thus providing valuable information for diagnosing or monitoring such diseases as diabetes and adrenal insufficiency. Indicator systems of the present invention which have two recognition elements are especially useful for detecting glucose in solutions which may also contain potentially interfering amounts of α-hydroxy acids or β-diketones (see co-pending application Ser. Nos. 09/754,217, filed Jan. 5, 2001; 60/329,746 filed Oct. 18, 2001; and 60/269,887 filed Feb. 21, 2001, entitled “Detection of Glucose in Solutions Also Containing An Alpha-Hydroxy Acid or a Beta-Diketone”, incorporated by reference). Medical/pharmaceutical production of glucose for human therapeutic application requires monitoring and control.

Uses for the present invention in agriculture include detecting levels of an analyte such as glucose in soybeans and other agricultural products. Glucose must be carefully monitored in critical harvest decisions for such high value products as wine grapes. As glucose is the most expensive carbon source and feedstock in fermentation processes, glucose monitoring for optimum reactor feed rate control is important in power alcohol production. Reactor mixing and control of glucose concentration also is critical to quality control during production of soft drinks and fermented beverages, which consumes the largest amounts of glucose and fermentable (cis-diol) sugars internationally.

When the detection system incorporates fluorescent indicator substituents, various detection techniques also are known in the art that can make use of the systems of the present invention. For example, the systems of the invention can be used in fluorescent sensing devices (e.g., U.S. Pat. No. 5,517,313) or can be bound to polymeric material such as test paper for visual inspection. This latter technique would permit, for example, glucose measurement in a manner analogous to determining pH with a strip of litmus paper. The systems described herein may also be utilized as simple reagents with standard benchtop analytical instrumentation such as spectrofluorometers or clinical analyzers as made by Shimadzu, Hitachi, Jasco, Beckman and others. These molecules would also provide analyte specific chemical/optical signal transduction for fiber optic-based sensors and analytical fluorometers as made by Ocean Optics (Dunedin, Fla.), or Oriel Optics.

U.S. Pat. No. 5,517,313, the disclosure of which is incorporated herein by reference, describes a fluorescence sensing device in which the systems of the present invention can be used to determine the presence or concentration of an analyte such as glucose or other cis-diol compound in a liquid medium. The sensing device comprises a layered array of a fluorescent indicator system-containing matrix (hereafter “fluorescent matrix”), a high-pass filter and a photodetector. In this device, a light source, preferably a light-emitting diode (“LED”), is located at least partially within the indicator material, or in a waveguide upon which the indicator matrix is disposed, such that incident light from the light source causes the indicator system to fluoresce. The high-pass filter allows emitted light to reach the photodetector, while filtering out scattered incident light from the light source.

The fluorescence of the indicator molecules employed in the device described in U.S. Pat. No. 5,517,313 is modulated, e.g., attenuated or enhanced, by the local presence of an analyte such as glucose or other cis-diol compound.

In the sensor described in U.S. Pat. No. 5,517,313, the material which contains the indicator is permeable to the analyte. Thus, the analyte can diffuse into the material from the surrounding test medium, thereby affecting the fluorescence emitted by the indicator system. The light source, indicator system-containing material, high-pass filter and photodetector are configured such that at least a portion of the fluorescence emitted by the indicator system impacts the photodetector, generating an electrical signal which is indicative of the concentration of the analyte (e.g., glucose) in the surrounding medium.

In accordance with other possible embodiments for using the indicator systems of the present invention, sensing devices also are described in U.S. Pat. Nos. 5,910,661, 5,917,605 and 5,894,351, all incorporated herein by reference.

The systems of the present invention can also be used in an implantable device, for example to continuously monitor an analyte in vivo (such as blood glucose levels). Suitable devices are described in, for example, co-pending U.S. patent application Ser. No. 09/383,148 filed Aug. 26, 1999, as well as U.S. Pat. Nos. 5,833,603, 6,002,954 and 6,011,984, all incorporated herein by reference.

The systems of the present invention can be prepared by persons skilled in the art without an undue amount of experimentation using readily known reaction mechanisms and reagents, including reaction mechanisms which are consistent with the general procedures described below.

### EXAMPLE 1

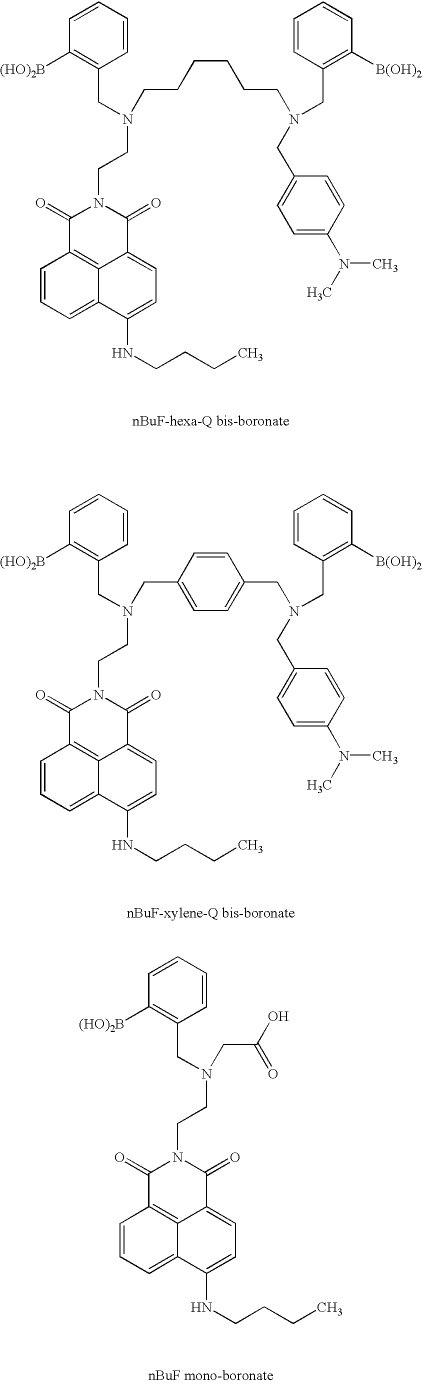

### N-2-[5-(N-4-dimethylaminobenzyl)-5-[2-(borono)benzyl]-aminohexyl]-[2-(borono)benzyl]aminoethyl-4-butylamino-1,8-naphthalimide (nBuF-hexa-Q bis-boronate)

The free bis boronic acid product used in glucose studies results from dissolution of N-2-[5-(N-4-dimethylaminobenzyl)-5-[2-(5,5-dimethylborinan-2-yl)benzyl]aminohexyl]-[2-(5,5-dimethylborinan-2-yl)benzyl]aminoethyl-4-butylamino-1,8-naphthalimide in the MeOH/PBS buffer system.

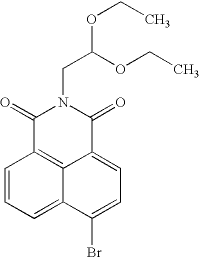

### N-(2,2-diethoxyethyl)-4-bromo-1,8-naphthalimide

A suspension of 4-bromo-1,8-naphthalic anhydride (10.0 g, 36.1 mmol) and aminoacetaldehyde diethyl acetal (4.81 g, 5.26 mL, 36.1 mmol, 1 equiv.) in 45 mL EtOH was stirred at 45 C for 3 days. At this time, the resulting suspension was filtered, washed with EtOH and the residue was dried to yield 13.3 g (94%) of a light brown solid product.

TLC: Merck silica gel 60 plates plates, Rf 0.17 with 98/2 CH₂Cl₂/CH₃OH, see with UV (254/366).

HPLC: HP 1100 HPLC chromatograph,Waters 5×100 mm NovaPak HR C18 column, 0.050 mL injection, 0.75 mL/min, 1.5 mL injection loop, 360 nm detection, A=water (0.1% HFBA) and B=MeCN (0.1% HFBA), gradient 10% B 2 min, 10-80% B over 18 min, 80-100% B over 2 min, 100% B 2 min, retention time 24.2 min.

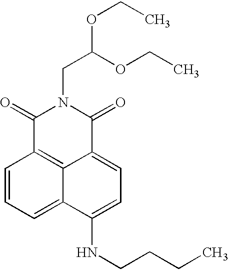

N- (2, 2-diethoxyethyl) -4-butylamino-1 ,8-naphthalimide

A solution of N- (2, 2-diethoxyethyl) -4-bromo-1, 8-naphthalimide (0.797 g, 2.03 mmol) and n-butylamine (1.48 g, 2.00 mL, 20.2 mmol, 9.96 equiv.) in 8 mL NMP was heated at 45 C for 66 hours. At this time, the resulting suspension was allowed to cool to 25 C, followed by filtration. The residue was dissolved with 50 mL ether and extracted 3×50 mL water. The organic extract was dried over anhydrous Na₂SO₄, filtered and concentrated to yield a crude yellow powder. The crude material was purified by silica gel chromatography (25 g gravity grade gel, 0-1% CH₃OH/CH₂Cl₂) to yield 0.639 g (82%) of a yellow powder.

TLC: Merck silica gel 60 plates, Rf 0.71 with 95/5 CH₂Cl₂/CH₃OH, see with UV (254/366).

HPLC: HP 1100 HPLC chromatograph, Waters 5×100 mm NovaPak HR C18 column, 0.050 mL injection, 0.75 mL/min, 1.5 mL injection loop, 450 nm detection, A=water (0.1% HFBA) and B=MeCN (0.1% HFBA), gradient 10% B 2 min, 10-80% B over 18 min, 80-100% B over 2 min, 100% B 2 min, retention time 23.5 min.

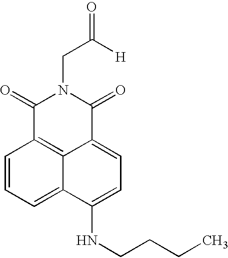

### N- (2-oxoethyl) -4-butylamino-1,8-naphthalimide

A solution of N-(2,2-diethoxyethyl)-4-butylamino-1,8-naphthalimide (0.622 g, 1.62 mmol) and p-toluenesulfonic acid mono hydrate (0.010 g, 0.053 mmol, 0.032 equiv.) in 25 mL acetone was stirred at 25 C for 18 hours. At this time, the solution was concentrated and the residue purified by silica gel chromatography (25 g gravity grade gel, 0-1% CH₃OH/CH₂Cl₂) to yield 0.470 g (94%) of an orange solid.

TLC: Merck silica gel 60 plates, Rf 0.61 with 95/5 CH₂Cl₂/CH₃OH, see with UV (254/366).

¹H NMR (400 MHz, CDCl₃); δ 1.03 (t, 3H, J=7.3 Hz) , 1.53 (m, 2H), 1.78 (m, 2H), 3.38 (t, 2H, J=7.2 Hz), 5.02 (s, 2H), 6.64 (d, 1H, J=8.6 Hz), 7.52 (dd, 1H, J=7.4, 8.3 Hz), 8.08 (dd, 1H, J=1 Hz, 8.5 Hz), 8.38 (d, 1H, J=8.3 Hz), 8.46 (dd, 1 H, J=1.0, 7.3 Hz), 9.75 (s, 1H)

HPLC: HP 1100 HPLC chromatograph, Waters 5×100 mm NovaPak HR C18 column, 0.050 mL injection, 0.75 mL/min, 1.5 mL injection loop, 450 nm detection, A=water (0.1% HFBA) and B=MeCN (0.1% HFBA), gradient 10% B 2 min, 10-80% B over 18 min, 80-100% B over 2 min, 100 %B 2 min, retention time 19.6 min.

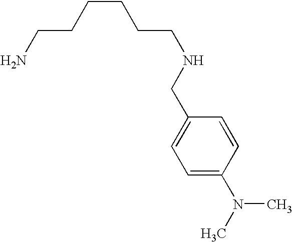

### N-(4-dimethylaminobenzyl)-1,6-diaminohexane

A suspension of 4-dimethylaminobenzaldehyde (1.00 g, 6.70 mmol), Na₂SO₄ (6.70 g, 47.2 mmol, 7.04 equiv.) and 1,6-diaminohexane (3.89 g, 33.5 mmol, 5.00 equiv.) in 20 mL anhydrous EtOH was stirred in the dark at 25 C under an atmosphere of nitrogen gas for 18 hours. At this time, the solution was filtered and NaBH₄ (1.73 g, 45.8 mmol, 6.84 equiv.) was added to the filtrate. The suspension was stirred at 25 C for 5 hours. At this time, the reaction mixture was concentrated and the residue dissolved in 50 mL water and extracted in 3 ×50 mL ether. The combined organic extracts were washed in 2×50 mL water. The combined aqueous extracts were extracted in 2×50 mL ether. The combined organic extracts were dried over Na₂SO₄, filtered and concentrated to yield 1.35 g (81%) of a viscous oil.

TLC: Merck silica gel 60 plates, Rf 0.58 with 80/15/5 CH₂Cl₂/CH₃OH/iPrNH₂, see with ninhydrin stain, UV (254/366).

HPLC: HP 1100 HPLC chromatograph, Waters 5×100 mm NovaPak HR C18 column, 0.050 mL injection, 0.75 mL/min, 1.5 mL injection loop, 280 nm detection, A=water (0.1% HFBA) and B=MeCN (0.1% HFBA), gradient 10% B 2 min, 10-80% B over 18 min, 80-100% B over 2 min, 100% B 2 min, retention time 13.3 min.

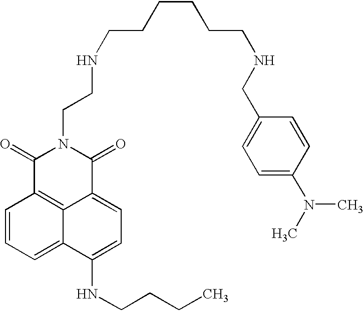

### N-2-[5-(N-4-dimethylaminobenzyl)aminohexyl]aminoethyl)-4-butylamino-1,8-naphthalimide

To a suspension of N-(2-oxoethyl)-4- butylamino -1,8-naphthalimide (0.346 g, 1.11 mmol) in 25 mL anhydrous MeOH was added a solution of N-(4-dimethylaminobenzyl)-1,6-diaminohexane (0.554 g, 2.22 mmol, 2.00 equiv.) and acetic acid (0.067 g, 1.1 mmol, 1.0 equiv.) in 20 mL anhydrous MeOH. To this mixture was added a solution of NaCNBH₃ (0.070 g, 1.1 mmol, 1.0 equiv.) in 5 mL anhydrous MeOH. The reaction mixture was stirred at 25C for 15 hours. At this time, the MeOH was removed by rotary evaporation and the residue was dissolved in 30 mL water. The solution was adjusted to pH 2 with 1 N HCl and then stirred for 1 hour at 25C. At this time, the solution was adjusted to pH 12 with 1 N NaOH and subsequently extracted in 3×50 mL CH₂Cl₂. The combined organic extracts were washed in 3×50 mL water, dried over anhydrous Na₂SO₄, filtered and concentrated to yield a crude brown oil. The crude material was purified by silica gel chromatography (35 g flash grade gel, 0-50% CH₃OH/CH₂Cl₂, then 45/50/5 CH₃OH/CH₂Cl₂/iPrNH₂ ) to yield 0.190 g (32%) of diamine product.

FAB MS: Calc'd for C₃₃H₄₅N₅O₂ [M]⁺ 544; Found [M]⁺ 544.

TLC: Merck silica gel 60 plates, Rf 0.42 with 80/20 CH₂Cl₂/CH₃OH, see with ninhydrin stain and UV (254/366).

HPLC: HP 1100 HPLC chromatograph, Waters 5×100 mm NovaPak HR C18 column, 0.050 mL injection, 0.75 mL/min, 1.5 mL injection loop, 450 nm detection, A=water (0.1% HFBA) and B=MeCN (0.1% HFBA), gradient 10% B 2 min, 10-80% B over 18 min, 80-100% B over 2 min, 100% B 2 min, retention time 17.6 min.

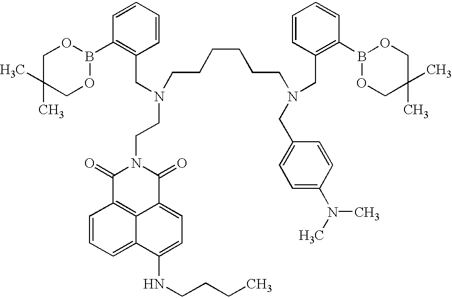

### N-2-[5-(N-4-dimethylaminobenzyl)-5-[2-(5,5-dimethylborinan-2-yl)benzyl)benzyl]aminohexyl]-[2-(5,5-dimethylborinan-2-yl)benzyl]aminoethyl-4-butylamino-1,8-naphthalimide

To a solution of N-2-[5-(N-4-dimethylamino-benzyl)aminohexyl]aminoethyl)-4-butylamino-1,8-naphthalimide (0.150 g, 0.276 mmole) and DIEA (0.355 g, 0.478 mL, 2.81 mmole, 10.0 equiv.) in 5 mL CHCl₃ was added a solution of (2-bromomethylphenyl)boronic acid neopentyl ester (0.390 g, 1.38 mmole, 5.00 equiv.) in 2 mL CHCl₃. The solution was subsequently stirred at 25C for 27 hours. At this time, the mixture was concentrated and the residue was purified by alumina column chromatography (100 g activated neutral alumina, 0-5% CH₃OH/CH₂Cl₂) to yield 0.024 g (19%) of a viscous brown oil.

FAB MS (glycerol matrix) : Calc'd for C₅₃H₆₇B₂N₅O₈[M]+ 924 (bis glycerol adduct in place of bis neopentyl ester of boronic acids); Found [M]+ 924

TLC: Merck neutral alumina plates, Rf 0.62 with 80/20 CH₂Cl₂/CH₃OH, see with UV (254/366).

HPLC: HP 1100 HPLC chromatograph, Waters 5×100 mm NovaPak HR C18 column, 0.050 mL injection, 0.75 mL/min, 1.5 mL injection loop, 450 nm detection, A=water (0.1% HFBA) and B=MeCN (0.1% HFBA), gradient 10% B 2 min, 10-80% B over 18 min, 80-100% B over 2 min, 100% B 2 min, retention time 20.7 min.

nBuF-xylene-Q bis-boronate:

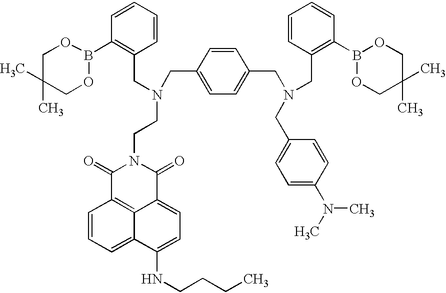

### N-2-[4-(N-4-dimethylaminobenzyl)-[2-(borono)benzyl]amino-methyl]benzyl-[2-(borono)benzyl]aminoethyl-4-butylamino-1,8-naphthalimide (nBuF-xylene-Q bis-boronate)

This compound is prepared in an analogous fashion to N-2-[5-(N-4-dimethylaminobenzyl)-5-[2-(borono)benzyl]-aminohexyl]-[2-(borono)benzyl]aminoethyl-4-butylamino-1,8-naphthalimide (nBuF-hexa-Q-bis boronate), using 1-[N-(4-dimethylaminobenzyl)amino]methyl-4-aminomethylbenzene as the diamine coupling partner.

### Control Indicator Molecule

nBuF mono-boronate:

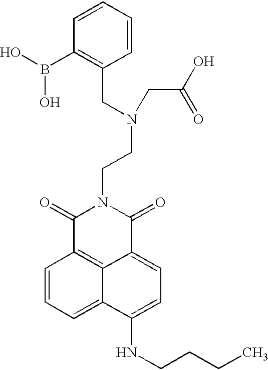

### N-2-(carboxymethyl)-2-[2-(borono)benzyl]aminoethyl-4-butylamino-1,8-naphthalimide (nBuF mono-boronate)

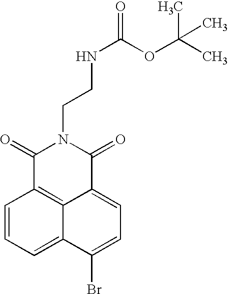

### N-2- (tert-butoxycarbonyl) aminoethyl-4-bromo-1 ,8-naphthalimide

A suspension of 4-bromo-1,8-naphthalic anhydride (1.00 g, 3.61 mmol) and N-(tert-butoxycarbonyl)-1,2-diaminoethane (0.578 g, 3.61 mmol, 1.00 equiv.) in 20 mL EtOH was stirred at 45 C for 2 hours. At this time, the temperature was ramped to 150 C over a 15 minute period.

Subsequently, the reaction mixture was cooled to 25 C and stirred for a further 15 hours. At this time, the resulting suspension was filtered, washing with EtOH and the residue was dried to yield 1.03 g (68%) of a light brown solid product.

TLC: Merck silica gel 60 plates plates, Rf 0.63 with 95/5 CH₂Cl₂/CH₃OH, see with UV (254/366).

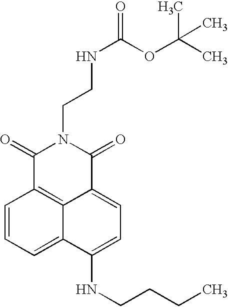

### N-2-(tert-butoxycarbonyl)aminoethyl-4-butylamino-1,8-naphthalimide

A solution of N-2-(tert-butoxycarbonyl)aminoethyl-4-bromo-1,8-naphthalimide (0.900 g, 2.15 mmol) and n-butylamine (0.786 g, 1.06 mL, 10.7 mmol, 5.01 equiv.) in 5 mL NMP was heated at 45 C for 17 hours. At this time, a second portion of n-butylamine (0.786 g, 1.06 mL, 10.7 mmol, 5.01 equiv.) was added. The resulting solution was stirred at 25 C for 23 hours longer. At this time, the mixture was concentrated in vacuo. The residue was purified by silica gel chromatography (50 g gravity grade gel, 0%, then 4% CH₃OH/CH₂Cl₂ step gradient) to yield 0.97 g of a sticky yellow solid containing residual NMP. The material was carried on as is.

FAB MS: Calc'd for C₂₃H₂₉N₃O₄ [M]+ 411; Found [M]+411.

TLC: Merck silica gel 60 plates, Rf 0.5 with 95/5 CH₂Cl₂/CH₃OH, see with UV (254/366).

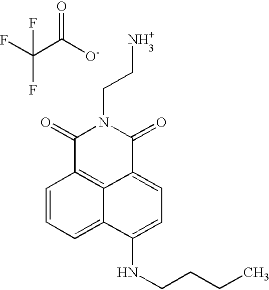

### N-2-aminoethyl-4-butylamino-1,8-naphthalimide mono TFA salt

A solution of N-2-(tert-butoxycarbonyl)aminoethyl-4-bromo-1,8-naphthalimide (0.92 g, 2.24 mmol) in 20 mL of 20% trifluoroacetic acid/CH₂Cl₂ was stirred at 25 C for 19 hours. At this time, the reaction mixture was concentrated under a stream of nitrogen gas. The residue was triturated using ether and the resulting solid was dried in vacuo to yield 0.772 g (81%) of an orange powder.

FAB MS: Calc'd for C₁₈H₂₁N₃O₂ [M]⁺ 311; Found [M+1]hu + 312.

HPLC: HP 1100 HPLC chromatograph, Vydac 201TP 10×250 mm column, 0.100 mL injection, 2 mL/min, 450 nm detection, A=water (0.1% HFBA) and B=MeCN (0.1% HFBA), gradient 10% B 2 min, 10-80% B over 18 min, 80-100% B over 2 min, 100% B 2 min, retention time 19.5 min.

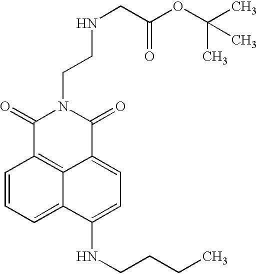

### N-2-[(tert-butoxycarbonyl)methyl]aminoethyl-4-butylamino-1,8-naphthalimide

A solution of N-2-aminoethyl-4-butylamino-1,8-naphthalimide mono TFA salt (0.99 g, 0.23 mmol), DIEA (0.167 g, 0.225 mL, 1.29 mmol, 5.55 equiv.) and tert-butyl bromoacetate (0.032 g, 0.024 mL, 0.16 mmol, 0.70 equiv.) in 2.5 mL of CH₂Cl₂ was stirred at 25 C for 23 hours. At this time, 25 mL CH₂Cl₂, were added, the solution was washed with 1×25 mL saturated NaHCO₃, the organic extract was dried over anhydrous Na₂SO₄, filtered and concentrated. The residue was purified by silica gel chromatography (15 g gravity grade gel, 0%-4% CH₃OH/CH₂Cl₂) to yield 0.051 g (73%) of a yellow glassy solid

TLC: Merck silica gel 60 plates, Rf 0.27 with 95/5 CH₂Cl₂/CH₃OH, see with UV (254/366).

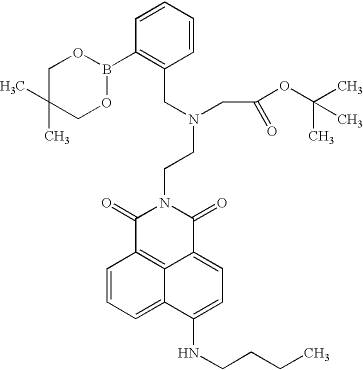

### N-2-[(tert-butoxycarbonyl)methyl]-2-[2-(5,5-dimethylborinan-2-yl)benzyl]aminoethyl-4-butylamino-1,8-naphthalimide

A solution of N-2-[(tert-butoxycarbonyl)methyl]-aminoethyl-4-butylamino-1,8-naphthalimide (0.0.051 g, 0.0.12 mmole), DIEA (0.78 g, 0.11 mL, 0.60 mmole, 5.0 equiv.) and (2-bromomethylphenyl)boronic acid neopentyl ester (0.083 g, 0.29 mmole, 2.4 equiv.) in 10 mL CH₂Cl₂ was stirred at 25° C. for 72 hours. At this time, the mixture was concentrated and purified by silica gel chromatography (10 g gravity grade gel, 0-1% CH₃OH/CH₂Cl₂) to yield 0.035 g (47%) of a glassy orange solid. The product was carried on as is.

TLC: Merck silica gel 60 plates, Rf 0.39 with 95/5 CH₂Cl₂/CH₃OH, see with UV (254/366).

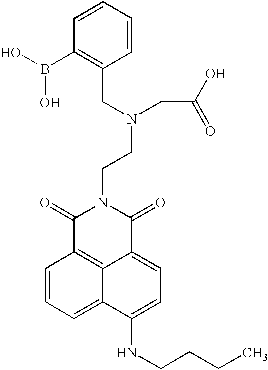

### N-2-(carboxymethyl)-2-[2-(borono)benzyl]aminoethyl-4-butylamino-1,8-naphthalimide (nBuF mono-boronate)

A solution of N-2-[(tert-butoxycarbonyl)methyl]-2-[2-(5,5-dimethylborinan-2-yl)benzyl]aminoethyl-4-butylamino-1,8-naphthalimide (0.035 g, 0.056 mmol) in 5 mL of 20% TFA/CH₂Cl₂ was stirred at 25C for 16 hours. At this time, the solution was concentrated under a stream of nitrogen gas and the residue was triturated with ether to yield an orange solid. The crude material was purified by silica gel chromatography (8 g gravity grade gel, 0-5% CH₃OH/CH₂Cl₂) to yield 0.011 g (39%) of a yellow/orange solid.

FAB MS: Calc'd for C₃₀H₃₄BN₃O₇ [M]+ 559 (mono glycerol adduct); Found [M+1]⁺ 560.

TLC: Merck silica gel 60 plates, Rf 0.26 with 95/5 CH₂Cl₂/CH₃OH, see with UV (254/366).

### Modulation of Fluorescence

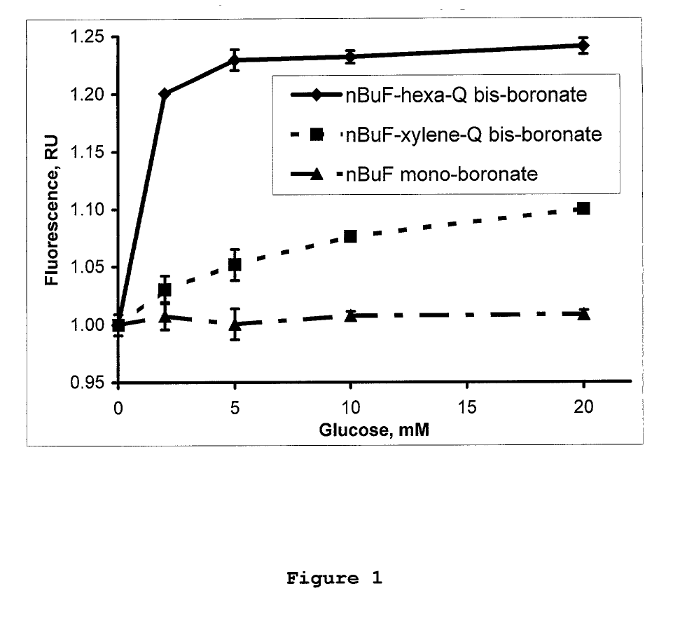

The modulation by glucose of the fluorescence of three compounds prepared in this example was determined. FIG. 1 shows the normalized fluorescence emission (I/Io @ 535 nm) of solutions of nBuF-hexa-Q bis-boronate (“hexa-Q”) indicator (0.015 mM), nBuF-xylene-Q bis-boronate (“xylene Q”) indicator (0.049 mM) and nBuF mono-boronate control indicator (0.029 mM) in 70/30 MeOH/PBS containing 0-20 mM glucose. Spectra were recorded using a Shimadzu RF-5301 spectrafluorometer with excitation @ 450 nm; excitation slits at 1.5 nm; emission slits at 1.5 nm; ambient temperature. Error bars are standard deviation with triplicate values for each data point.

The data show that the fluorescence of the nBuF mono-boronate indicator compound is unaffected by the presence of glucose. The fluorescence of the nBuF-xylene-Q bis-boronate indicator compound is marginally affected by glucose, and the fluorescence of the nBuF-hexa-Q bis-boronate indicator compound is greatly affected by glucose in the range of 0-5 mM. It is believed that in the absence of glucose, the relatively flexible hexamethylene linkage in the hexa-Q compound allows the N-4-dimethylaminobenzyl quenching group to be sufficiently close to the naphthalimide fluorophore to effectively quench the latter'fluorescence. In the presence of glucose, both boronic acid recognition elements would be expected to participate in glucose binding, thus changing the indicator'molecular configuration and sufficiently separating the fluorophore and quencher such that the fluorescent emission is dequenched. The same effect is seen with the xylene-Q compound, but to a much lesser degree since the xylene linker is less flexible, thus permitting less separation between the fluorophore and quencher upon glucose binding.

The control compound contains a fluorophore group but no quencher. The control emits fluorescence in the absence of glucose, which is not modulated when glucose is added.

### EXAMPLE 2

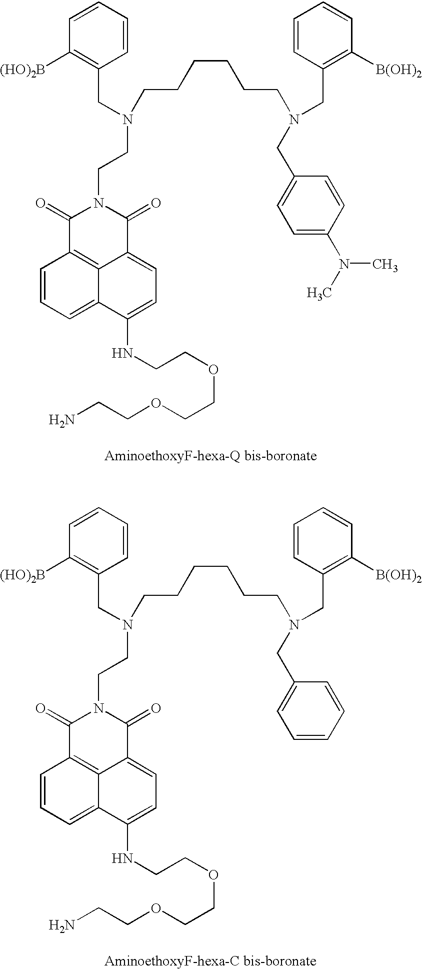

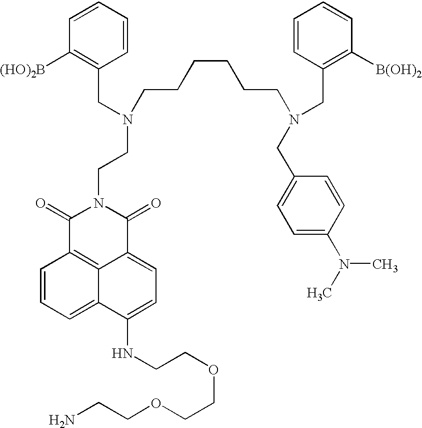

### N-2-[5-(N-4-dimethylaminobenzyl)-5-[2-(borono)benzyl]-aminohexyl]-[2-(borono)benzyl]aminoethyl-4-[2-(2-aminoethoxy)ethoxyethyl)amino-1,8-naphthalimide (aminoethoxyF-hexa-Q bis-boronate)

This compound was prepared in an analogous fashion to N-2-[5-(N-4-dimethylaminobenzyl)-5-[2-(borono)benzyl]aminohexyl]-[2-(borono)benzyl]aminoethyl-4-butylamino-1,8-naphthalimide (nBuF-hexa-Q bis-boronate) with the following modification. The 4-bromo position of the 1,8-naphthalimide moiety was not converted to the 2-(2-aminoethoxy)ethoxyethyl)amino group until after the bis benzylboronation of the diamine intermediate was complete. This final step was carried out by the addition of 2,2′-(ethylenedioxy)bis(ethylamine) to the bromide under similar conditions for the addition of butyl amine in the synthesis of N-(2,2-diethoxyethyl)-4-butylamino-1,8-naphthalimide.

aminoethoxyF-hexa-C bis-boronate:

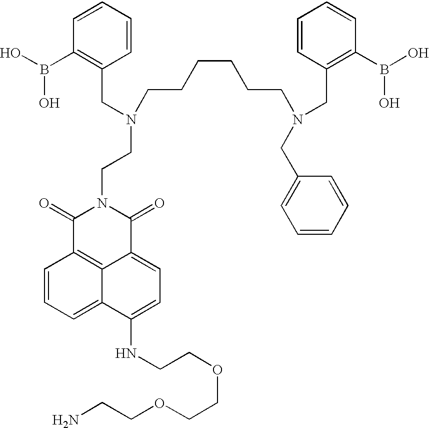

### N-2-[5-benzyl-5-[2-(borono)benzyl]aminohexyl]-[2-(borono)benzyl]aminoethyl-4-[2-(2-aminoethoxy) ethoxyethyl) amino-1, 8-naphthalimide (aminoethoxyF-hexa-C bis-boronate)

This compound was prepared in an analogous fashion to N-2-[5-(N-4-dimethylaminobenzyl) -5-[2-(borono)benzyl]aminohexyl]-[2-(borono) benzyl]aminoethyl-4-[2-(2-aminoethoxy) ethoxyethyl) amino-1,8-naphthalimide (aminoethoxyF-hexa-Q bis-boronate), using N-benzyl-1,6-diaminohexane as the diamine coupling partner.

### Modulation of Fluorescence

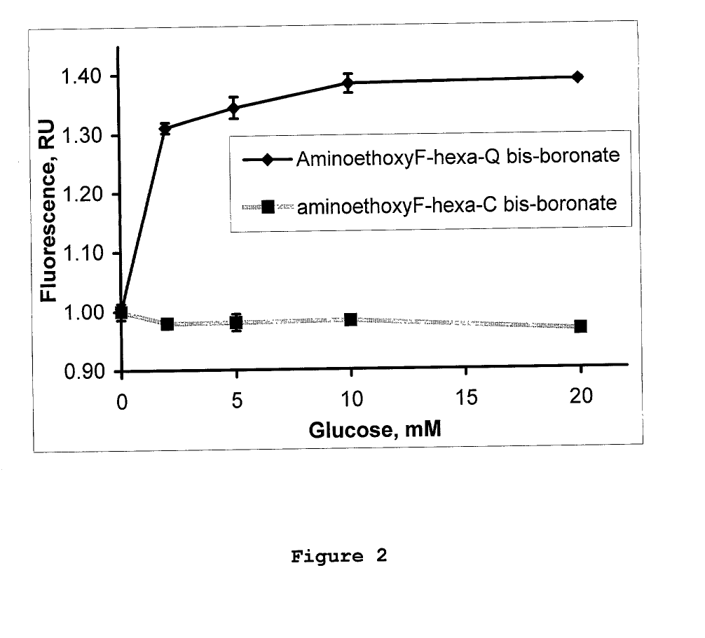

The modulation by glucose of the fluorescence of the two compounds prepared in this example was determined. FIG. 2 shows the normalized fluorescence emission (I/Io @535 nm) of solutions of aminoethoxyF-hexa-Q-bis boronate indicator (0.197 mM) and aminoethoxyF-hexa-C-bis boronate control indicator in 70/30 MeOH/PBS containing 0-20 mM glucose. Spectra were recorded using a Shimadzu RF-5301 spectrafluorometer with excitation @ 450 nm; excitation slits at 1.5 nm; emission slits at 1.5 nm; ambient temperature. Error bars are standard deviation with duplicate values for each data point.

The data show that the fluorescence of the hexa-C indicator compound is unaffected by the presence of glucose, and the fluorescence of the hexa-Q indicator compound is greatly affected by glucose in the range of 0-10 mM. It is believed that in the absence of glucose, the relatively flexible hexamethylene linkage in the hexa-Q compound allows the N-4-dimethylaminobenzyl quenching group to be sufficiently close to the naphthalimide fluorophore to effectively quench the latter'fluorescence. In the presence of glucose, both boronic acid recognition elements would be expected to participate in glucose binding, thus changing the indicator'molecular configuration and sufficiently separating the fluorophore and quencher such that the fluorescent emission is dequenched.

The hexa-C compound is identical to the hexa-Q compound, but lacks the dimethylamino group needed for effective quenching of the naphthalimide fluorophore. The hexa-C compound emits fluorescence in the absence of glucose, which is not modulated when glucose is added.

The following Examples 3-5 illustrate a glucose sensing approach where the indicator system contains a boronic acid recognition element and a catechol ligand element. The general principle of this approach can be illustrated by the following formula:

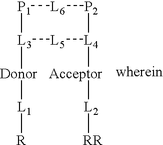

Donor is a fluorophore, and Acceptor is a fluorophore or a quencher;

Donor and Acceptor are selected such that energy from Donor can be transferred to Acceptor in a molecular distance dependent manner;

L₁, L₂ L₃, and L₄ are independently chemical linkers with from about 3 to about 20 contiguous atoms and comprised by, but not limited to, the following substituted or/and non-substituted chemical groups (aliphatic, aromatic, amino, amide, sulfo, carbonyl, ketone, sulfonamide, etc.);

R is a glucose recognition element comprising one or two phenylboronic acid groups;

RR is a chemical group capable of forming a reversible ester bond with phenylboronic acid derivatives of R, for example, an aromatic diol (e.g., a catechol), lactate, α-hydroxy acids, tartaric acid, malic acid, glucose, diethanolamine, polyhydroxy vicinal diols (all optionally substituted), etc.;

L₃₋₆and P₁₋₂ are optional groups and may be present independently;

L₅ and L₆ are linking groups as defined for linking groups L₁₋₄, or polymer chains comprised of, for example, acrylamides, acrylates, polyglycols, or other hydrophilic polymers; and

P₁ and P₂ are hydrophilic or hydrophobic polymers.

When R and RR are allowed to interact in free solution, or when suitably immobilized on a hydrophilic polymer, Donor and Acceptor are disposed sufficiently close to each other to allow relatively efficient energy transfer from the Donor to Acceptor (for example, via FRET, collisional energy transfer, etc.) . When glucose is added to the solution it competes with RR for the binding of R(boronate) leading to the shift in the RR-R ⇄ RR+R equilibrium to the right. When free in solution or when immobilized using relatively long and flexible linkers on the polymer, the R-Donor and RR-Acceptor moieties can move away from each other and the energy transfer efficiency between the Donor and Acceptor is reduced, resulting in increased fluorescent emission.

### EXAMPLE 3

### Effect of glucose on fluorescence emission of N-(5-methoxycarbonyl-5-[3,4-dihydroxybenzamido]pentyl)-N′-(5-fluoresceinyl)thiourea (fluorescein-catechol adduct) in phospate buffered saline in the presence of N-α-(3-boronato-5-nitro)benzoyl-N-ε-(4-dimethylamino-3,5-dinitro)benzoyllysine (quencher-boronic acid adduct)

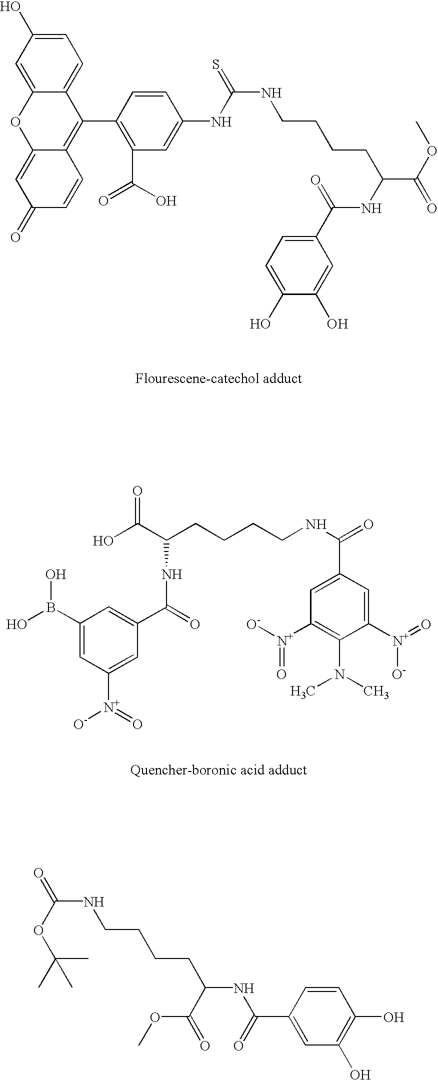

### N-α-(3,4-dihydroxybenzoyl)-N-ε-t-BOC-lysine methyl ester

3,4-dihydroxybenzoic acid (820 mg, 5.3 mmole) and N-ε-t-BOC-lysine methyl ester (1.38 g, 5.31 mmole) were dissolved in 50 mL EtOAc/THF (1/1, anhydrous). Dicyclohexylcarbodiimde (1.24 g, 6 mmole) was added to the solution. The reaction mixture was stirred for 24 hours, filtered, and the solvent was evaporated. The solid obtained was dissolved in EtOAc (50 mL) and extracted with phosphate buffer (200 mM, pH=6.5) 2×50 mL. The ethyl acetate solution was washed with brine, separated, dried with Na₂SO₄, and evaporated to produce 1.89 g of solid (90% yield). The compound was pure by TLC and used as is for the next step.

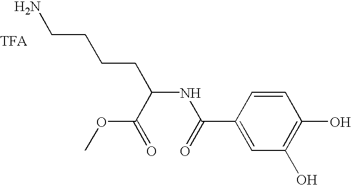

N-α-(3,4-dihydroxybenzoyl)-lysine methyl ester trifluoroacetate salt

N-α-(3,4-dihydroxybenzoyl)-N-ε-t-BOC-lysine methyl ester (840 mg, 2.12 mmole) was combined with 10 mL of CH₂Cl₂, 3 mL of trifluoroacetic acid, and 1 mL of triisopropylsilane. After stirring overnight at room temperature, the solution was evaporated, the resulting residue was washed with ether, and dried under vacuum. Yield 808 mg (93%).

HPLC: HP 1100 HPLC chromatograph, Waters 5×100 mm NovaPak HR C18 column, 0.100 mL injection, 0.75 mL/min, 2 mL injection loop, 370 nm detection, A=water (0.1% HFBA) and B=MeCN (0.1% HFBA), gradient 10% B 2 min, 10-80% B over 18 min, 80-100% B over 2 min, 100% B 2 min, retention time 10.78 min.

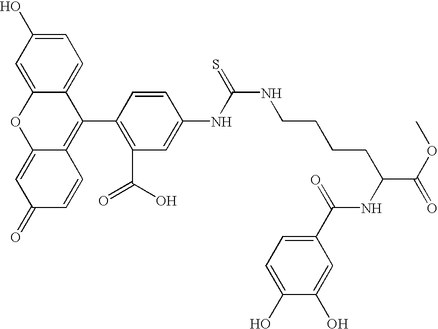

### N-(5-methoxycarbonyl-5-[3,4-dihydroxybenzamido]pentyl)-N′-(5-fluoresceinyl)thiourea

N-α-(3,4-dihydroxybenzoyl)-lysine methyl ester trifluoroacetate salt (60 mg, 0.146 mmole), fluorescein isothiocyanate (50 mg, 0.128 mmole), and diisopropylethylamine (129 mg, 1 mmole) were combined with 1 mL of anhydrous DMF. The reaction was stirred for 5 hours followed by evaporation of the solvent. The residue was subjected to chromatography on SiO₂ (10 g) with CH₂Cl₂/MeOH (80/20 by vol.) as eluent. Isolated product-68 mg, (77 % yield).

FAB MS: Calculated for C₃₅H₃₁N₃O₁₀S: M=685; Found M+1=686. HPLC: HP 1100 HPLC chromatograph, Waters 5×100 mm NovaPak HR C18 column, 0.100 mL injection, 0.75 mL/min, 2 mL injection loop, 370 nm detection, A=water (0.1% HFBA) and B=MeCN (0.1% HFBA), gradient 10% B 2 min, 10-80% B over 18 min, 80-100% B over 2 min, 100% B 2 min, retention time 16.59 min.

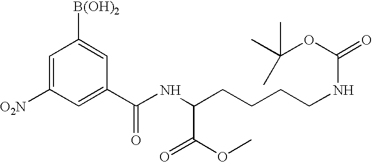

### N-α-(3-boronato-5-nitro)benzoyl-N-ε-t-BOC-lysine methyl ester

(3-carboxy-5-nitrophenyl)boronic acid (536 mg, 2.54 mmole), N-ε-t-BOC-lysine methyl ester hydrochloride (776 mg, 2.61 mmole), and diphenylphosphoryl azide (718 mg, 2.6 mmole) were combined with 5 mL of anhydrous DMF. Diisopropylethylamine (1.3 mL, 7.5 mmole) was added to the DMF solution. The solution was stirred at room temperature for 24 hours. DMF was evaporated in vacuum, the residue was dissolved in 50 mL of EtOAc, and the EtOAc solution was extracted with H₂O (3×50 mL). After an extraction with brine, the organic phase was separated, dried with Na₂SO₄, and the solvent was evaporated to produce 880 mg of product (76% yield). Product was carried on as is.

HPLC: HP 1100 HPLC chromatograph, Waters 5×100 mm NovaPak HR C18 column, 0.050 mL injection, 0.75 mL/min, 1.5 mL injection loop, 450 nm detection, A=water (0.1% HFBA) and B=MeCN (0.1% HFBA), gradient 10% B 2 min, 10-80% B over 18 min, 80-100% B over 2 min, 100% B 2 min, retention time 17.87 min.

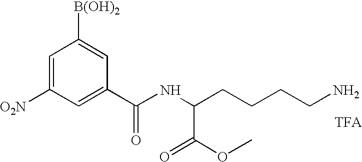

### N-α-(3-boronato-5-nitro)benzoyl-lysine methyl ester trifluoroacetate salt

N-α-(3-boronato-5-nitro)benzoyl-N-ε-t-BOC-lysine methyl ester (800 mg, 1.76 mmole) was combined with 10 mL of CH₂Cl₂, 3 mL of trifluoroacetic acid, and 1 mL of triisopropylsilane. After stirring overnight at room temperature, the solution was evaporated, the resulting residue was washed with ether, and dried under vacuum. Yield 715 mg (87%). Product was carried on as is.

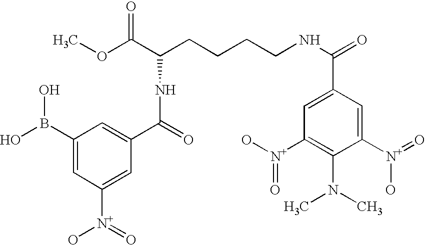

### N-α-(3-boronato-5-nitro)benzoyl-N-ε-(4-dimethylamino-3,5-dinitro) benzoyllysine methyl ester

A solution of N-α-(3-boronato-5-nitro)benzoyl-lysine methyl ester trifluoroacetate salt (0.198 g, 0.42 mmole), DIEA (0.167 g, 0.225 mL, 1.29 mmole, 3.05 equiv.), 4-dimethylamino-3,5-dinitrobenzoic acid (0.120 g, 0.47 mmol, 1.11 equiv.) and diphenylphosphorylazide (0.130 g, 0.47 mmole, 1.11 equiv.) in 3 mL DMF at 25 C was stirred in the dark for 23 hours. At this time, 50 mL EtOAc were added and the solution was washed in 2×20 mL portions of 100 mM phosphate buffer (pH 6.5), then 1×25 mL NaCl (sat'd aqueous solution). The organic extract was dried over anhydrous Na₂SO₄, filtered and concentrated to yield crude orange solid. The residue was purified by silica gel column chromatography (10 g gravity grade gel, 0-5% CH₃OH/CH₂Cl₂) to yield 0.0974 g (39%) of a yellow-orange solid. Product was carried on as is.

TLC: Merck silica gel 60 plates, Rf 0.60 with 80/20 CH₂Cl₂/CH₃OH, see with UV (254/366)

HPLC: HP 1100 HPLC chromatograph, Waters 5×100 mm NovaPak HR C18 column, 0.050 mL injection, 0.75 mL/min, 1.5 mL injection loop, 450 nm detection, A=water (0.1% HFBA) and B=MeCN (0.1% HFBA), gradient 10% B 2 min, 10-80% B over 18 min, 80-100% B over 2 min, 100% B 2 min, retention time 18.91 min.

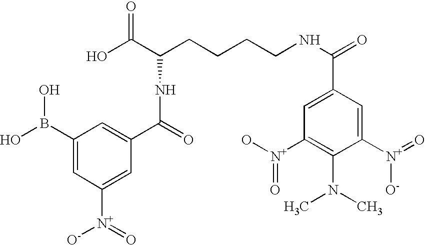

### N-α-(3-boronato-5-nitro)benzoyl-N-ε-(4-dimethylamino-3,5-dinitro)benzoyllysine

A solution of N-α-(3-boronato-5-nitro)benzoyl-N-ε-(4-dimethylamino-3,5-dinitro)benzoyllysine methyl ester (0.095 g, 0.16 mmole) in 4 mL of 1:1 Na₂CO₃ (0.2 M aqueous):EtOH was stirred at 25 C for 1 hour, then 45 C for 1.5 hours. At this time, the mixture was concentrated in vacuo, followed by the addition of 25 mL of 5% TFA/CH₂Cl₂. The mixture was washed 2×10 mL water, followed by the addition of 25 mL more 5% TFA/CH₂Cl₂ to the organic layer. The organic extract was dried over anhydrous Na₂SO₄, filtered and concentrated to yield 0.088 g (95%) of an orange powder.

FAB MS: Glycerol matrix; Calc'd for C₂₅H₂₉BN₆O₁₃ (mono glycerol adduct) [M]+ 632; Found [M+1]⁺ 633.

HPLC: HP 1100 HPLC chromatograph, Waters 5×100 mm NovaPak HR C18 column, 0.050 mL injection, 0.75 mL/min, 1.5 mL injection loop, 450 nm detection, A=water (0.1% HFBA) and B=MeCN (0.1% HFBA), gradient 10% B 2 min, 10-80% B over 18 min, 80-100% B over 2 min, 100% B 2 min, retention time 17.66 min.

### Fluorescent Modulation

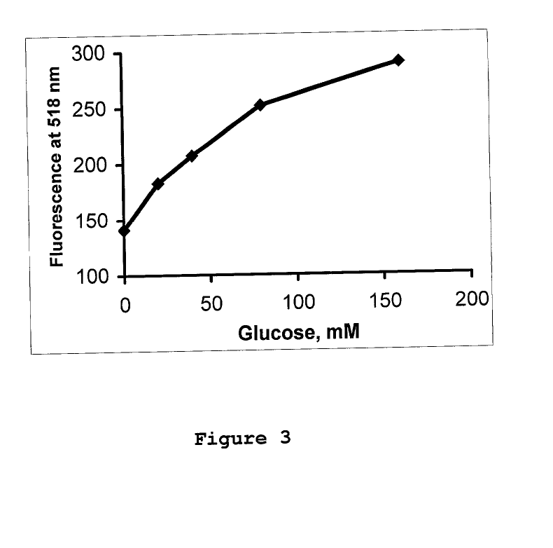

FIG. 3 shows the fluorescence emission (I at 518 nm) of a 2 μM solution of the fluorescein-catechol adduct in PBS containing 30 μM of quencher-boronic acid adduct. The concentration of glucose was varied from 0-160 mM. Spectra were recorded using a Shimadzu RF-5301 spectrafluorometer with excitation at 495 nm; excitation slits at 3 nm; emission slits at 5 nm; low PMT sensitivity, ambient temperature. The quenching decreased with addition of glucose.

### EXAMPLE 4

### Effect of glucose on fluorescence emission of N-α-(3,4-dihydroxybenzoyl)-N-ε-(5-dimethylaminonaphthalene-1-sulfonyl)-lysine (DANSYL-catechol adduct) in phospate buffered saline in the presence of N-α-(3-boronato-5-nitro)benzoyl-Nε-(4-dimethylamino-3,5-dinitro)benzoyl-lysine (quencher-boronic acid adduct)

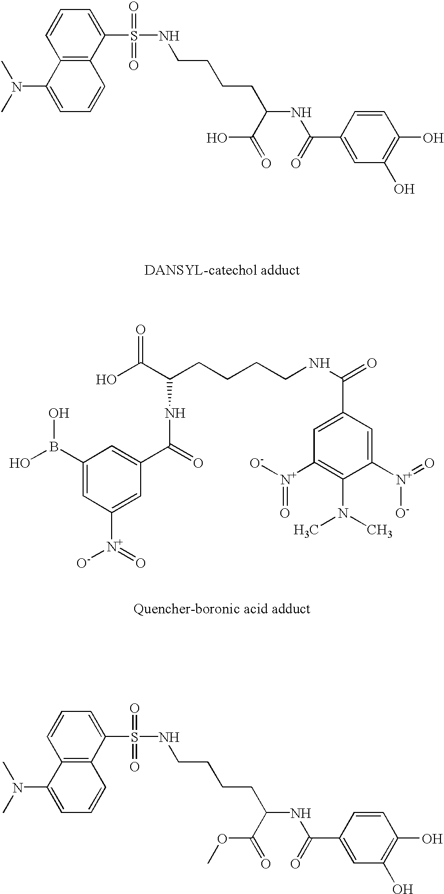

### N-α-(3,4-dihydroxybenzoyl)-N-ε-(5-dimethylamino-naphthalene-1-sulfonyl) -lysine methyl ester

N-α-(3,4-dihydroxybenzoyl)-lysine methyl ester trifluoroacetate salt (205 mg, 0.5 mmole, see example 3 for synthesis) and DANSYL chloride (162 mg, 06 mmole) were combined with 2 mL of anhydrous DMF. Diisopropylethylamine (224 mg, 1.7 mmole) was added to the DMF solution. The solution was stirred at room temperature for 5 hours followed by evaporation of DMF in vacuum. The residue was subjected to silica gel chromatography (CH₂Cl₂/MeOH, 98/2 by vol.). The product was obtained as a yellow solid—240 mg (90% yield).

FAB MS: Calculated for C₂₉H₃₁N₃O₇S: M=529; Found M+1=530.

HPLC: HP 1100 HPLC chromatograph, Waters 5×100 mm NovaPak HR C18 column, 0.100 mL injection, 0.75 mL/min, 2 mL injection loop, 370 nm detection, A=water (0.1% HFBA) and B=MeCN (0.1% HFBA), gradient 10% B 2 min, 10-80% B over 18 min, 80-100% B over 2 min, 100% B 2 min, retention time 15.45 minutes.

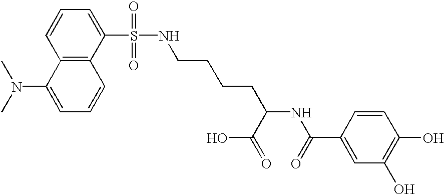

### N-α-(3,4-dihydroxybenzoyl)-N-ε-(5-dimethylamino-naphthalene-1-sulfonyl)-lysine

N-α-(3,4-dihydroxybenzoyl)-N-ε-(5-dimethylamino-naphthalene-1-sulfonyl)-lysine methyl ester (200 mg, 0.38 mmole) and 250 mg of Na₂CO₃ were combined with 10 mL of EtOH/H₂O (1/1 by vol.). The mixture was stirred at 55° C. for 6 hours. The solvent was evaporated in vacuum and 1 mL of trifluoroacetic acid was added to neutralize excess base, 50 mL of EtOAc was added to the mixture and the solution was extracted with H₂O (2×40 mL) . The organic phase was separated, dried with Na₂SO₄, and evaporated to yield 190 mg of solid (97% yield).

HPLC: HP 1100 HPLC chromatograph, Waters 5×100 mm NovaPak HR C18 column, 0.100 mL injection, 0.75 mL/min, 2 mL injection loop, 370 nm detection, A=water (0.1% HFBA) and B=MeCN (0.1% HFBA), gradient 10% B 2 min, 10-80% B over 18 min, 80-100% B over 2 min, 100% B 2 min, retention time 14.26 min.

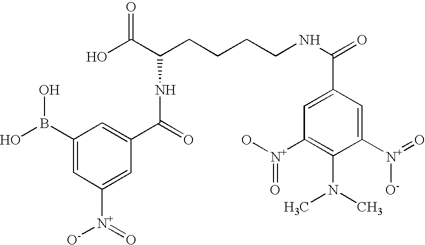

### N-α-(3-boronato-5-nitro)benzoyl-N-ε-(4-dimethylamino-3,5-dinitro)benzoyllysine

See example 3 for synthesis.

### Fluorescent Modulation

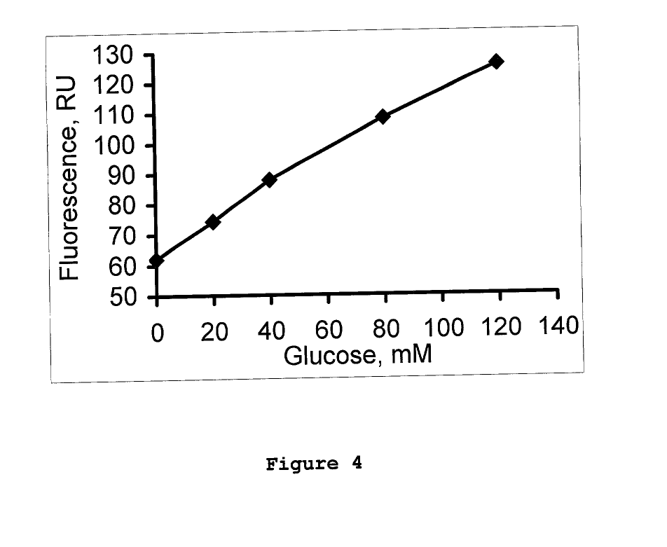

FIG. 4 shows the fluorescence emission (I at 545 nm) of a 30 μM solution of the DANSYL-catechol adduct in PBS containing 120 μM of quencher-boronic acid adduct. The concentration of glucose was varied from 0-120 mM. Spectra were recorded using a Shimadzu RF-5301 spectrafluorometer with excitation at 350 nm; excitation slits at 3 nm; emission slits at 5 nm; high PMT sensitivity, ambient temperature. The quenching decreased with addition of glucose.

### EXAMPLE 5

### Effect of glucose on fluorescence emission of acrylamide gel containing N-α-(3,4-dihydroxybenzoyl)-N-ε-(5-dimethylaminonaphthalene-1-sulfonyl)-lysine N-3-(methacrylamido)propylcarboxamide (DANSYL-catechol monomer) and N-α-(3-boronato-5-nitro)benzoyl-N-ε-(4-dimethylamino-3,5-dinitro)benzoyllysine N-3-(methacrylamido)propylcarboxamide (quencher-boronic acid monomer).

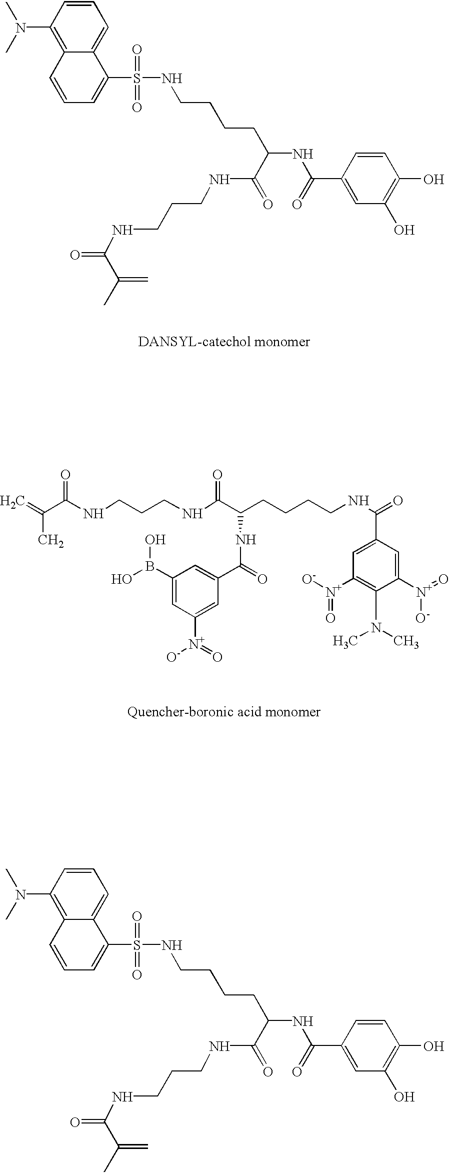

### N-α-(3,4-dihydroxybenzoyl)-N-ε-(5-dimethylamino-naphthalene-1-sulfonyl)-lysine N-3-(methacrylamido)-propylcarboxamide

N-α-(3,4-dihydroxybenzoyl)-N-ε-(5-dimethylamino-naphthalene-1-sulfonyl)-lysine (75 mg, 0.15 mmole; for synthesis see example 4), 3-aminopropylmethacrylamide hydrochloride salt (30 mg, 0.17 mmole), diisopropylethylamine (0.1 mL, 0.5 mmole), and 2 mL of anhydrous DMF were combined. 1-[3-(dimethylamino)-propyl]-3-ethylcarbodiimide hydrochloride (40 mg, 0.2 mmole) was dissolved in 2 mL of anhydrous CH₂Cl₂. The DMF and CH₂Cl₂ solutions were combined and stirred at room temperature for 20 hours. The solvent was evaporated in vacuum and the residue was subjected to SiO₂ (7 g) chromatography producing 18 mg of product (19% yield).

FAB MS: Calculated for C₃₂H₄₁N₅O₇S: M=640; Found M+=640.

HPLC: HP 1100 HPLC chromatograph, Waters 5×100 mm NovaPak HR C18 column, 0.100 mL injection, 0.75 mL/min, 2 mL injection loop, 370 nm detection, A=water (0.1% HFBA) and B=MeCN (0.1% HFBA), gradient 10% B 2 min, 10-80% B over 18 min, 80-100% B over 2 min, 100% B 2 min, retention time 14.78 min.

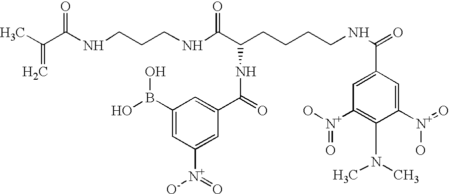

### N-α-(3-boronato-5-nitro)benzoyl-N-ε-(4-dimethylamino-3,5-dinitro)benzoyllysine N-3-(methacrylamido)propyl-carboxamide

A solution of 3-aminopropylmethacrylamide hydrochloride salt (0.013 g, 0.073 mmole, 1.2 equiv.), DIEA (0.025 g, 0.034 mL, 0.19 mmole, 3.2 equiv.), N-α-(3-boronato-5-nitro)benzoyl-N-ε-(4-dimethylamino-3,5-dinitro)benzoyllysine (0.035 g, 0.061 mmole; for synthesis see example 3), diphenylphosphorylazide (0.019 g, 0.015 mL, 0.069 mmole, 1.1 equiv.) and ˜2 mg of BHT in 1 mL anhydrous DMF at 25 C was stirred in the dark for 23.5 hours. At this time, 60 mL EtOAc were added and the solution was washed in 2×20 mL portions of 200 mM phosphate buffer (pH 6.5), then 1×20 mL NaCl (sat'd aqueous solution). The organic extract was dried over anhydrous Na₂SO₄, filtered and concentrated to yield an orange solid. The solid was triturated with ether and dried to yield 0.028 g (65%) of an orange powder.

FAB MS: Glycerol matrix; Calc'd for C₃₂H₄₁BN₈O₁₃ (mono glycerol adduct) [M]⁺ 756; Found [M+1]⁺ 757.

HPLC: HP 1100 HPLC chromatograph, Waters 5×100 mm NovaPak HR C18 column, 0.050 mL injection, 0.75 mL/min, 1.5 mL injection loop, 450 nm detection, A=water (0.1% HFBA) and B=MeCN (0.1% HFBA), gradient 10% B 2 min, 10-80% B over 18 min, 80-100% B over 2 min, 100% B 2 min, retention time 17.98 min.

### Preparation of acrylamide gel (20%) containing N-α-(3,4-dihydroxybenzoyl)-N-ε-(5-dimethylaminonaphthalene-1-sulfonyl)lysine N-3-(methacrylamido)propylcarboxamide and N-α-(3-boronato-5-nitro)benzoyl-Nε-(4-dimethylamino-3,5-dinitro)benzoyllysine N-3-(methacrylamido)propyl-carboxamide

A solution of acrylamide (20% wt.) and N,N′-methylenebisacrylamide (0.6% wt.) in ethylene glycol was prepared. N-α-(3,4-dihydroxybenzoyl)-N-ε-(5-dimethylaminonaphthalene-1-sulfonyl)-lysine N-3-(methacrylamido)propylcarboxamide (0.75 mg, 1.6×10⁻⁶ mole), N-α-(3-boronato-5-nitro)benzoyl-N-ε-(4-dimethylamino-3,5-dinitro)benzoyllysine N-3-(methacrylamido)propylcarboxamide (3.5 mg, 5×10⁻⁶ mole), and 30 μL of aqueous ammonium persulfate (5% wt) were combined with 0.5 mL of ethylene glycol monomer solution. The resulting solution was placed in a glove box purged with nitrogen. An aqueous solution of N,N,N′,N′-tetrametylethylenediamine (30 μL, 5% wt.) was added to the monomer formulation to accelerate polymerization. The resulting formulation was poured in a mold constructed from microscope slides and 100 μ stainless steel spacer. After being kept for 8 hours in a nitrogen atmosphere, the mold was placed in phosphate buffered saline (PBS) (10 mM PBS, pH=7.4), the microscope slides were separated, and the hydrogel was removed. The hydrogel was washed with 100 mL of PBS containing 1 mM lauryl sulfate sodium salt and 1 mM EDTA sodium salt for 3 days, the solution being changed every day, followed by washing with DMF/PBS (10/90 by vol., 3×100 mL), and finally with PBS (pH=7.4, 3×100 mL). The resulting hydrogel polymer was stored in PBS (10 mM PBS, pH=7.4) containing 0.2% wt. sodium azide and 1 mM EDTA sodium salt.

### Fluorescent Modulation

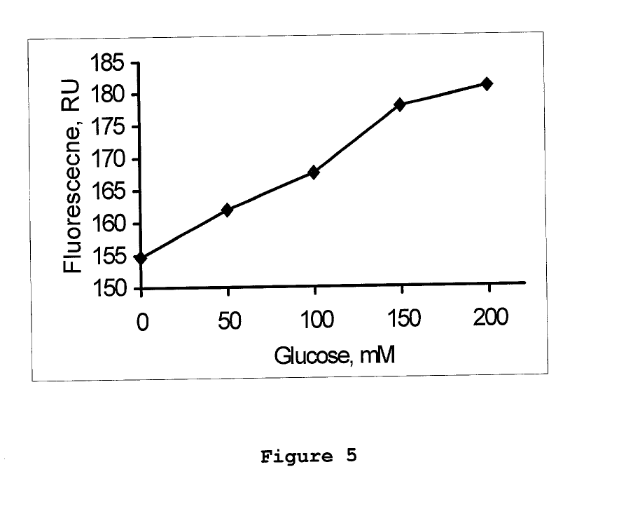

FIG. 5 shows the fluorescence emission (I at 532 nm) of an acrylamide gel (20%) containing 2 mM of the DANSYL-catechol monomer and 10 mM of quencher-boronic acid monomer in PBS. The gel (100 μm thickness) is mounted in a PMMA cuvette. The concentration of glucose was varied from 0-200 mM. Spectra were recorded using a Shimadzu RF-5301 spectrafluorometer with excitation at 350 nm; excitation slits at 3 nm; emission slits at 10 nm; high PMT sensitivity, 37° C. The quenching decreased with addition of glucose.

### EXAMPLE 6

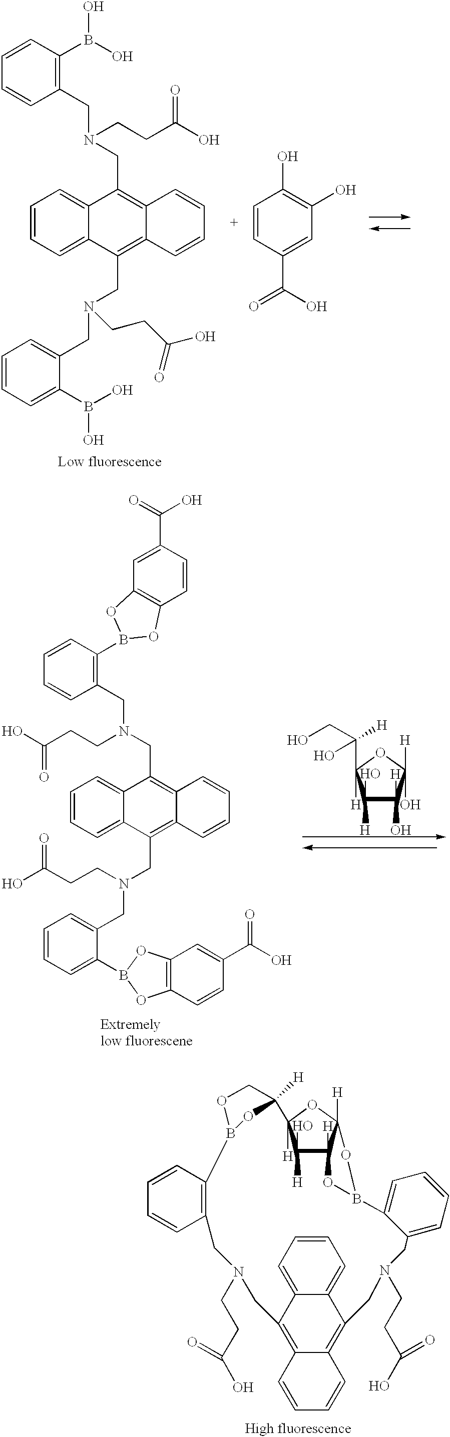

### Effect of glucose on fluorescence of anthracene bis-boronic acid derivative in the presence of 3,4-dihydroxy benzoic acid

### Preparation of PBS soluble anthracene bis boronic acid derivative

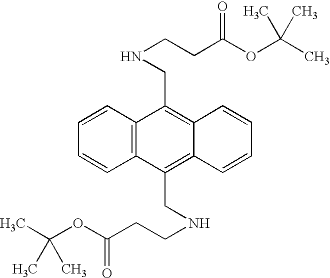

### 9,10-bis[[2-(tert-butoxycarbonyl)ethylamino]methyl]-anthracene

A solution of β-alanine tert-butyl ester hydrochloride (3.06 g, 16.8 mmole, 5.09 equiv.), DIEA (4.27 g, 5.75 mL, 33.0 mmole, 10.00 equiv.) and 9,10-bis(chloromethyl)anthracene (0.910 g, 3.31 mmole) in 75 mL CHCl₃ at 23° C. was stirred in the dark for 93 hours. At this time, the solution was filtered and washed with 1×40 mL and 2×60 mL portions of NaHCO₃ (sat'd aqueous solution). The organic extract was dried over anhydrous Na₂SO₄, filtered and concentrated to yield a crude yellow solid. The residue was purified by silica gel column chromatography (30 g gravity grade gel, 0-3% CH₃OH/CH₂Cl₂) to yield 1.06 g (65%) of a viscous yellow-orange. Product was carried on as is.

TLC: Merck silica gel 60 plates, Rf 0.33 with 95/5 CH₂Cl₂/CH₃OH, see with UV (254/366).

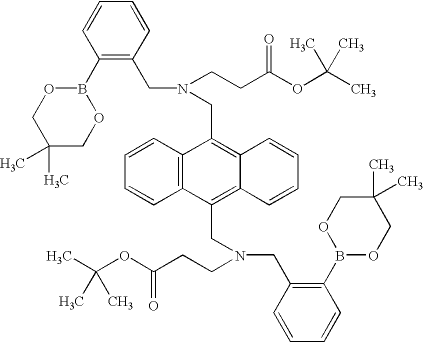

### 9,10-bis[N-[2-(5,5-dimethylborinan-2-yl)benzyl]-N-[2-(tert-butoxycarbonyl)ethylamino]methyl]anthracene

A solution of 9,10-bis[[2-(tert-butoxycarbonyl)-ethylamino]methyl]anthracene (1.60 g, 3.25 mmole), DIEA (4.45 g, 6.00 mL, 34.4 mmole, 10.6 equiv.) and (2-bromomethylphenyl)boronic acid neopentyl ester (4.80 g, 17.0 mmole, 5.22 equiv.) in 30 mL CHCl₃ at 23° C. was stirred in the dark for 4.5 days. At this time, 45 mL CHCl₃ were added to the mixture, and the mixture was washed with 2×25 mL portions of NaHCO₃ (sat'd aqueous solution). The organic extract was dried over anhydrous Na₂SO₄, filtered and concentrated to yield a crude reddish oil. The residue was purified by alumina column chromatography (100 g activated neutral alumina, 0-3% CH₃OH/CH₂Cl₂) to yield ˜3.5 g of an orange solid. The product was dissolved, followed by the formation of a white precipitate (DIEA-HBr salt). The solution was filtered and the filtrate concentrated to yield 2.72 g (93%) of an orange solid. Product (>80% pure by RP-HPLC) was carried on as is.

TLC: Merck basic alumina plates, Rf 0.66 with 95/5 CH₂Cl₂/CH₃OH, see with UV (254/366).

HPLC conditions: HP 1100 HPLC chromatograph, Vydac 201TP 10×250 mm column, 0.100 mL injection, 2 mL/min, 370 nm detection, A=water (0.1% HFBA) and B=MeCN (0.1% HFBA), gradient 10% B 2 min, 10-80% B over 18 min, 80-100% B over 2 min, 100% B 2 min, retention time 23.9 min.

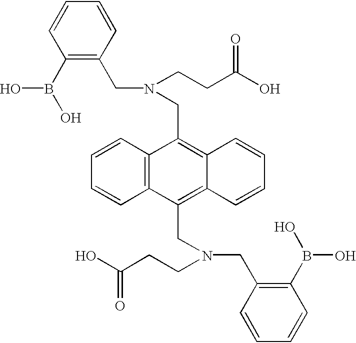

### 9,10-bis[N-(2-boronobenzyl)-N-[3-(propanoyl)amino]-methyl]anthracene

A solution of 9,10-bis[N-[2-(5,5-dimethylborinan-2-yl)benzyl]-N-[2-(tert-butoxycarbonyl)ethylamino]-methyl]anthracene (0.556 g, 0.620 mmole) in 5 mL 20% TFA/CH₂Cl₂ at 23° C. was stirred in the dark for 25 hours. At this time, the reaction mixture was concentrated under a stream of N₂ gas. The residue was triturated with 3×10 mL portions of ether. The residual solid was dried in vacuo to yield 0.351g (87%) of a fluffy yellow powder.

FAB MS: Glycerol matrix; Calc'd for C₄₂H₄₆B₂N₂O₁₀ (bis glycerol adduct) [M]⁺ 760; Found [M]⁺ 760.

HPLC: HP 1100 HPLC chromatograph, Waters 5×100 mm NovaPak HR C18 column, 0.025 mL injection, 0.75 mL/min, 1.5 mL injection loop, 360 nm detection, A=water (0.1% HFBA) and B=MeCN (0.1% HFBA), gradient 10% B 2 min, 10-80% B over 18 min, 80-100% B over 2 min, 100% B 2 min, retention time 16.7 min.

### Fluorescent Modulation

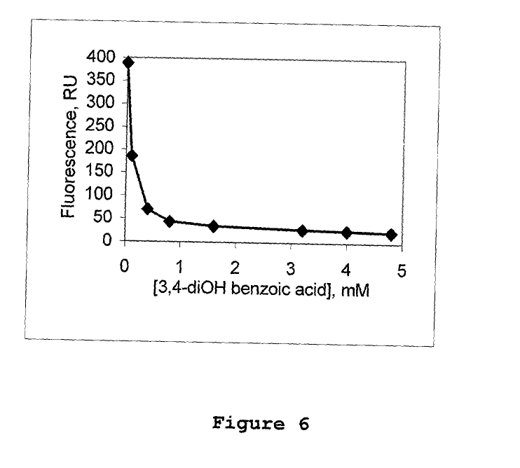

FIG. 6 shows the effect of 3,4-dihydroxybenzoic acid on fluorescence intensity (450 nm) of the anthracene bis boronic acid derivative (40 μM) in PBS prepared in this example. Spectra were recorded using a Shimadzu RF-5301 spectrafluorometer with excitation at 370 nm; excitation slits at 3 nm; emission slits at 3 nm; high PMT sensitivity, ambient temperature. The anthracene bis boronic acid derivative emits a low level of fluorescence, which is effectively quenched by the presence of 3,4-dihydroxybenzoic acid.

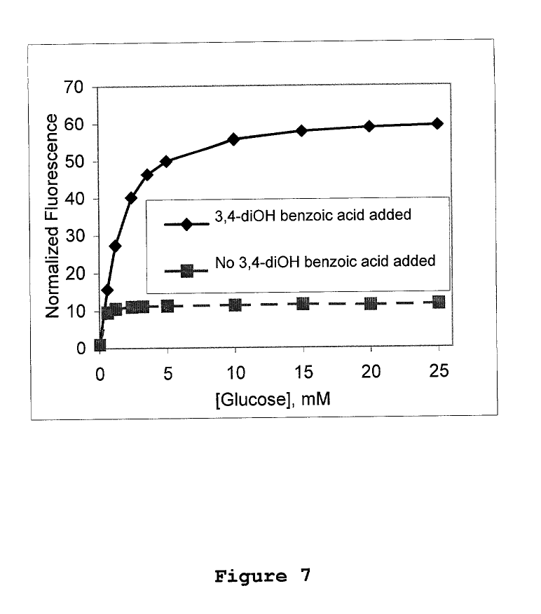

FIG. 7 shows the normalized fluorescence intensity (430 nm) of the anthracene bis boronic acid derivative (40 μM) of this example in the presence of 3,4-dihydroxybenzoic acid (200 μM) as a function of glucose concentration in PBS (diamonds as points), and the normalized fluorescence intensity (430 nm) of the same indicator (40 μM) as a function of glucose concentration in PBS (squares). The glucose concentration was varied from 0 to 25 mM. Spectra were recorded using a Shimadzu RF-5301 spectrafluorometer with excitation at 370 nm; excitation slits at 3 nm; emission slits at 5 nm; low PMT sensitivity, ambient temperature Addition of glucose to the anthracene bis boronic acid derivative in the absence of the 3,4-dihydroxybenzoic acid quencher results in an increase in fluorescence. Addition of glucose to the anthracene bis boronic acid derivative in the presence of the 3,4-dihydroxybenzoic acid quencher results in a marked increase in fluorescence. It is believed that the glucose displaces the 3,4-dihydroxybenzoic acid quencher from the boronic acid recognition element, resulting in increased fluorescence. In this example, the 3,4-dihydroxybenzoic acid group acts as both the quencher portion of the detection system, and as a ligand element interacting with the recognition element.

### EXAMPLE 7

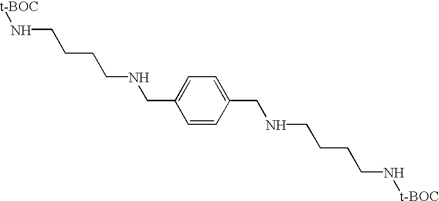

1,4-Bis[4-(tert-butoxycarbonyl) aminobutyl amino]methyl]benzene

Terephthaldicarboxaldehyde (0.253 g, 1.89 mmole), N-t-Boc-butanediamine (0.71 g, 3.77 mmole) and sodium sulfate (5.5 g, 40 mmole) were combined with 25 ml of anhydrous methanol. The mixture was stirred at room temperature for 24 hours, sodium sulfate was filtered off and NaBH₄ (1.5 g, 40 mmole) was added. After 4 hours the mixture was diluted with 100 ml of ether and filtered. The residue obtained after evaporation of the solvent was subjected to column chromatography on silica gel, CH₂Cl₂/MeOH/Et₃N (80/15/5 vol. %) as eluent. The product was isolated as a white solid (0.77 g, 86% yield) . This material was used as is in the next step.

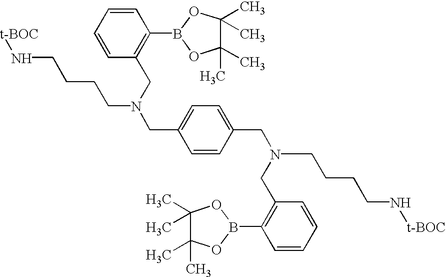

B. 1,4-Bis [N-[2-(pinacolato)boronobenzyl]-N-[[4-(tert-butoxycarbonyl)aminobutylamino]methyl]benzene

2-bromomethylphenyl boronic acid, pinacol ester (1.4 g, 4.7 mmole), 1,4-bis[[4-(tert-butoxycarbonyl)aminobutylamino]methyl]benzene (0.74 g, 1.56 mmole), and N,N-diisopropyl-N-ethylamine (1.8 ml, 10 mmole) were dissolved in 20 ml of CH₂Cl₂. The solution was stirred at room temperature for 24 hours, solvent was evaporated and the residue was washed with hexane/ether (50/50 vol., 3×10 ml). The product was further purified by column chromatography (SiO₂, 90/10 vol., CH₂Cl₂/MeOH). Yield 1.18 g (83%).

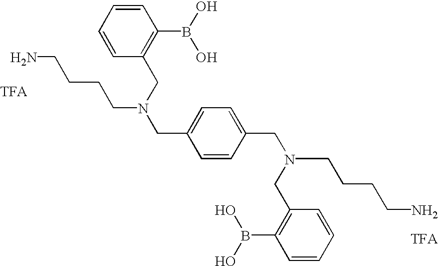

C. 1,4-Bis [N-(2-boronobenzyl)-N-[4-aminobutylamino]methyl]benzene bis trifluoroacetic acid salt

1,4-bis [N-[2-(pinacolato)boronobenzyl]-N-[[4-(tert-butoxycarbonyl)aminobutylamino]methyl]benzene (1.1 g, 1.2 mmole) was dissolved in 20 ml CH₂Cl₂ solution containing 20% vol. TFA and 5% vol. triisipropylsilane. The solution was stirred for 12 hours and the solvent was evaporated, the residue was dried under high vacuum at 50° C. for 24 hours. Yield quantitative. FAB MS: Calculated for C₄₂H₆₄B₂N₄O₄ M+=710 (bis pinacol ester), found M+2=712.

HPLC: HP 1100 HPLC chromatograph, Waters 5×100 mm NovaPak HR C18 column, 0.100 mL injection, 0.75 mL/min, 2 mL injection loop, 280 nm detection, A=water (0.1% HFBA) and B=MeCN (0.1% HFBA), gradient 10% B 2 min, 10-80% B over 18 min, 80-100% B over 2 min, 100% B 2 min, retention time 14.6 min.

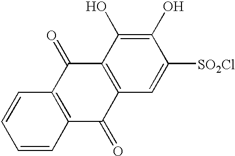

D. 3,4-Dihydroxy-9,10-dioxo-2-anthracenesulfonyl chloride

3,4-dihydroxy-9,10-dioxo-2-anthracenesulfonic acid sodium salt (1.4 g, 3.9 mM) was combined with 30 ml of chlorosulfonic acid and heated to 90° C. for 5 hours, after which the solution was cooled to 0° C. and poured into 100 g of ice. After the ice melted the solution was extracted with CH₂Cl₂ (3×100 ml), the methylene chloride extracts were combined, dried with Na₂SO₄ and evaporated to produce 0.87 g of solid (Yield 66%).

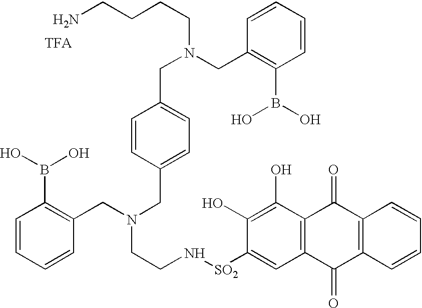

E. 1-[N-(2-Boronobenzyl)-N-[4-aminobutylamino]methyl]-4-[N-(2-boronobenzyl)-N-[4-[(3,4-dihydroxy-9,10-dioxo-2-anthracene)sulfonamido]butylamino)]methyl]-benzene trifluoroacetic acid salt

3,4-Dihydroxy-9,10-dioxo-2-anthracenesulfonyl chloride (0.095 g, 0.28 mmole) was dissolved in 3 ml of anhydrous CH₃CN and added dropwise to a solution of 1,4-bis [N-(2-boronobenzyl)-N-[4-aminobutylamino]methyl]benzene bis trifluoroacetic acid salt (1.06 g, 1.37 mmole) and N,N-diisopropyl-N-ethylamine (1 ml, 5.8 mmole) in 5 ml of anhydrous CH₃CN. After stirring for 4 hours the solvent was evaporated and the residue dried under high vacuum. The residue was dissolved in 10 ml of CH₃CN/TFA (80/20 vol. %) and the solvent was evaporated again. Water (10 ml) was added to the residue and the flask was sonicated for 20 minutes followed by filtration of the brown solid which contained the product. Further purification was achieved using preparative HPLC: HP 1100 HPLC chromatograph, Waters 25×100 mm NovaPak HR C18 column, 1.00 mL injection, 5 mL/min flow rate, 2 mL injection loop, 470 nm detection, A=water (0.1% HFBA) and B=MeCN (0.1% HFBA), gradient 10% B 2 min, 10-80% B over 18 min, 80-100% B over 2 min, 100% B 2 min, retention time 18.5 min. Yield: 198 mg (79%). This compound was tested for interaction with D-glucose in MeOH/PBS (1/1, vol.) solution, pH=7.4, interaction was evaluated by monitoring the absorbance spectra.

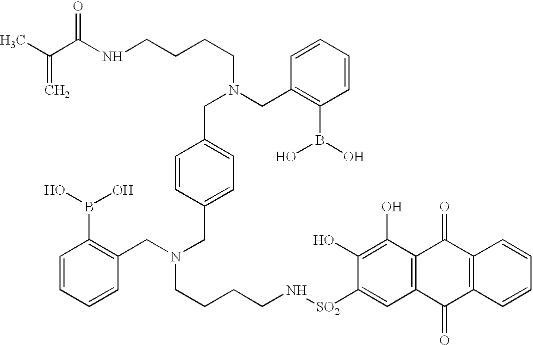

F. 1-[N-(2-Boronobenzyl)-N-[4-(methacrylamido)butylamino]methyl]-4-[N-(2-boronobenzyl)-N-[4-[(3,4-dihydroxy-9,10-dioxo-2-anthracene)sulfonamido]butylamino]methyl]-benzene

1-[N-(2-boronobenzyl)-N-[4-aminobutylamino]methyl]-4-[N-(2-boronobenzyl)-N-[4-[(3,4-dihydroxy-9,10-dioxo-2-anthracene)sulfonamido]butylamino]methyl]benzene trifluoroacetic acid salt (30 mg, 3.34×10^{31 5} mole) was dissolved in 1 ml of anhydrous MeOH. Methacrylic acid NHS ester (10 mg, 5.46×10^{31 5} mole, prepared according to J. Am. Chem. Soc., 1999, 121(15), 3617) was added followed by addition of 0.01 ml of Et₃N. The solution was stirred for 10 hours. The solvent was evaporated in vacuum and the solid was washed with H₂O. RP-HPLC analysis showed absence of starting material in the solid. The resulting solid was dried under vacuum and used as is for polymerization into a hydrogel film.

G. Preparation of N-N-dimethylacrylamide hydrogel film containing 1-[N-(2-boronobenzyl)-N-[4-(methacrylamido)butylamino]methyl]-4-[N-(2-boronobenzyl)-N-[4-[(3,4-dihydroxy-9,10-dioxo-2-anthracene)sulfonamido]butylamino]methyl]-benzene

A solution of N,N-dimethylacrylamide (40% wt.) and N,N′-methylenebisacrylamide (0.8% wt.) and D-fructose (200 mM) in DMF was prepared. 1-[N-(2-boronobenzyl)-N-[4-(methacrylamido)butylamino]methyl]-4-[N-(2-boronobenzyl)-N-[4-[(3,4-dihydroxy-9,10-dioxo-2-anthracene)sulfonamido]butylamino]methyl]-benzene (30 mg) was dissolved in 0.5 ml of DMF solution containing monomers and D-fructose. Aqueous ammonium persulfate (20 μL, 5% wt.) was combined with the formulation. The resulting solution was placed in a glove box purged with nitrogen. An aqueous solution of N,N,N′,N′-tetramethylethylenediamine (20 μL, 5% wt.) was added to the monomer formulation to accelerate polymerization. The resulting formulation was poured in a mold constructed from microscope slides and 100 μM stainless steel spacer. After being kept for 8 hours in a nitrogen atmosphere the mold was placed in phosphate buffered saline (10 mM pi, pH=7.4), the microscope slides were separated, and the hydrogel was removed. The hydrogel was washed with 100 ml of phosphate buffered saline (PBS) containing 1 mM lauryl sulfate sodium salt and 1 mM EDTA sodium salt for 3 days, the solution being changed every day, followed by washing with DMF/PBS (10/90 by vol., 3×100 ml), and finally with PBS (pH=7.4, 3×100 ml). The resulting hydrogel polymer was stored in PBS (10 mM PBS, pH=7.4) containing 0.2% wt. sodium azide and 1 mM EDTA sodium salt.

H. Effect of D-glucose and on fluorescence and absorbance of N,N-dimethylacrylamide gel containing l-[N-(2-boronobenzyl)-N-[4-(methacrylamido)butylamino]methyl]-4-[N-(2-boronobenzyl)-N-[4-[(3,4-dihydroxy-9,10-dioxo-2-anthracene)sulfonamido]butylamino]methyl]-benzene

This experiment was conducted in a Shimadzu RF-5301 PC spectrofluorimeter equipped with a variable temperature attachment. N,N-dimethylacrylamide hydrogel film was attached to a piece of a glass slide which was glued in a PMMA fluorescence cell at 450° angle. The cell was filled with PBS, pH=7.4, solutions containing various concentrations of D-glucose. The cell was equilibrated at 37° C. for 30 minutes prior to measurements of absorbance and fluorescence intensity. For fluorescence intensity measurements excitation wavelength was set at 470 nm, slit width was 3/3 nm, high sensitivity of PMT. The absorbance spectra of the hydrogel film were measured using an HP 8453 instrument, absorbance value at 690 nm was used for blank correction in each measurement.

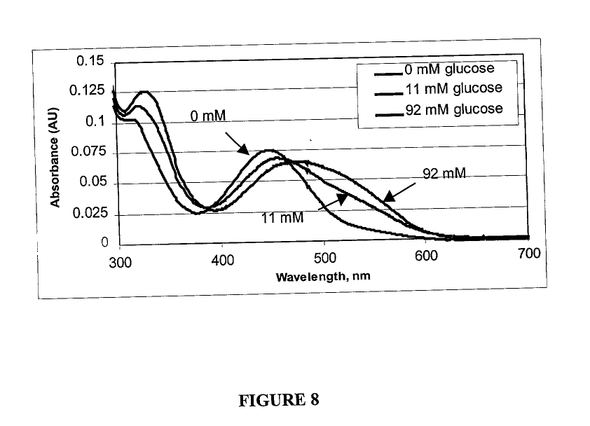

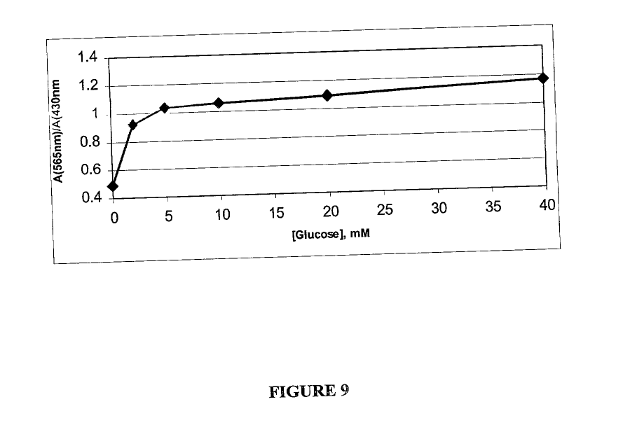

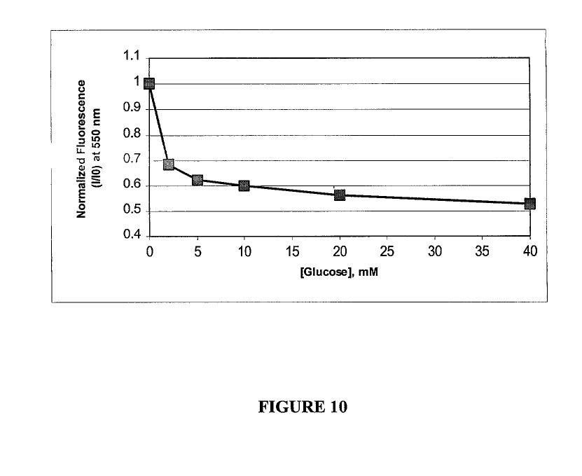

The results are shown in FIGS. 8-10. FIG. 8 shows the absorbance spectra of the indicator in PBS/methanol with varying concentrations of glucose. FIG. 9 shows the ratio of absorbance of the indicator gel (A (565 nm)/A (430 nm)) with various concentrations of glucose. FIG. 10 shows the normalized fluorescence (I/I₀) at 550 nm with various concentrations of glucose.

What is claimed is:

1. A method for detecting the presence or concentration of a polyhydroxyl analyte in a sample, which comprises:
a) exposing the sample to an indicator system having
i) a first recognition element capable of forming a covalent bond in a reversible fashion with said analyte, and either A) a second recognition element capable of forming a covalent bond in a reversible fashion to said analyte bound to the first recognition element, or B) a ligand element capable of interacting in a reversible fashion with the first recognition element in the absence of said analyte, said ligand element optionally further comprising a label that produces a detectable quality that is modulated by the interaction of the ligand element with the recognition element, wherein the portion of the indicator system containing said first recognition element is covalently or non-covalently linked to the portion of the indicator system containing said second recognition element or said ligand element; and
ii) a detection system which comprises at least one of A) a donor/acceptor system which produces a detectable quality that changes in a concentration-dependent manner when said indicator system is exposed to said analyte, or B) said labeled ligand element; and
b) measuring any change in said detectable quality to thereby determine the presence or concentration of said analyte in said sample.

2. The method of claim 1, wherein the indicator system has at least two recognition elements for the analyte.

3. The method of claim 2, wherein the analyte is a sugar and each recognition element is independently selected from the group consisting of boronic acid, boronate ion, arsenious acid, arsenite ion, telluric acid, tellurate ion, germanic acid, germanate ion, and combinations thereof.

4. The method of claim 3, wherein the analyte is glucose and each recognition element comprises one or more boronic acid groups.

5. The method of claim 1, wherein the indicator system has a recognition element for the analyte, and a ligand element.

6. The method of claim 5, wherein the analyte is a sugar, and the recognition element comprises one or more of the following: boronic acid, boronate ion, arsenious acid, arsenite ion, telluric acid, tellurate ion, germanic acid, or germanate ion.

7. The method of claim 6, wherein the analyte is glucose and the recognition element comprises one or more boronic acid groups.

8. The method of claim 5, wherein the ligand element is a moiety capable of forming an ester bond with the recognition element.

9. The method of claim 8, wherein the ligand element is selected from the group consisting of an aromatic diol, a lactate, an alpha-hydroxy acid, a tartaric acid, a malic acid, diethanolamine, a β-aminoalcohol, glucose, and a polyhydroxy compound, and a vicinal hydroxy-containing compound, all optionally substituted.

10. The method of claim 1, wherein the detection system comprises a donor/acceptor system.

11. The method of claim 10, wherein the detection system comprises a fluorophore and a quenching moiety, wherein said fluorophore is either quenched or dequenched when said indicator system binds to said analyte.

12. The method of claim 1, wherein the detection system comprises said labeled ligand element.

13. The method of claim 12, wherein said labeled ligand element comprises a fluorophore, and the fluorescence of said fluorophore is modulated by the binding of said indicator system with said analyte.

14. The method of claim 10, wherein the detection system comprises at least two different fluorophores, and wherein the fluorescence of said fluorophores is modulated by the interaction of said indicator system with said analyte.

15. The method of claim 1, wherein the sample is a physiological fluid.

16. The method of claim 15, wherein the physiological fluid is selected from the group consisting of blood, plasma, serum, interstitial fluid, cerebrospinal fluid, urine, saliva, intraocular fluid, lymph, tears, sweat, and physiological buffers.

17. The method of claim 1, wherein the indicator system is exposed to the sample in solution.

18. The method of claim 1, wherein the indicator system is immobilized on or within a solid support.

19. The method of claim 18, wherein the solid support is a polymeric matrix.

20. The method of claim 1, wherein the indicator system is associated with an implantable device, and wherein step a) takes place in vivo.

21. The method of claim 1, wherein the measuring step takes place at substantially ambient temperature.

22. The method of claim 21, wherein the temperature is up to about 80° C.

23. The method of claim 1, wherein the indicator system comprises a residue of a compound selected from the group consisting of:
N-2-[5-(N-4-dimethylaminobenzyl)-5-[2-(borono)-benzyl]aminohexyl]-[2-(borono)benzyl]aminoethyl-4-butylamino-1,8-naphthalimide;
N-2-[4-(N-4-dimethylaminobenzyl)-[2-(borono)-benzyl]aminomethyl]benzyl-[2-(borono)benzyl]aminoethyl-4-butylamino-1,8-naphthalimide;
N-2-[5-(N-4-dimethylaminobenzyl)-5-[2-(borono)-benzyl]aminohexyl]-[2-(borono)benzyl]aminoethyl-4-[2-(2-aminoethoxy)ethoxyethyl)amino-1,8-naphthalimide;
N-(5-methoxycarbonyl-5-[3,4-dihydroxybenz-amido]pentyl)-N′-(5-fluoresceinyl)thiourea;
N-α-(3-boronato-5-nitro)benzoyl-N-ε-(4-dimethylamino-3,5-dinitro)benzoyllysine;
N-α-(3,4-dihydroxybenzoyl)-N-ε-(5-dimethylaminonaphthalene-1-sulfonyl)-lysine;
N-α-(3,4-dihydroxybenzoyl)-N-ε-(5-dimethylaminonaphthalene-1-sulfonyl)-lysine N-3-(methacrylamido)propylcarboxamide; and
N-α-(3-boronato-5-nitro)benzoyl-N-ε-(4-dimethylamino-3,5-dinitro)benzoyllysine N-3-(methacrylamido)propyl -carboxamide.

24. An indicator system which comprises a residue of a compound selected from the group consisting of:
N-2-[5-(N-4-dimethylaminobenzyl)-5-[2-(borono)-benzyl]aminohexyl]-[2-(borono)benzyl]aminoethyl-4-butylamino-1,8-naphthalimide;
N-2-[4-(N-4-dimethylaminobenzyl)-[2-(borono)-benzyl]aminomethyl]benzyl-[2-(borono)benzyl]aminoethyl-4-butylamino-1,8-naphthalimide;
N-2-[5-(N-4-dimethylaminobenzyl)-5-[2-(borono)-benzyl]aminohexyl]-[2-(borono)benzyl]aminoethyl-4-[2-(2-aminoethoxy)ethoxyethyl)amino-1,8-naphthalimide;
N-(5-methoxycarbonyl-5-[3,4-dihydroxybenz-amido]pentyl)-N′-(5-fluoresceinyl)thiourea;
N-α-(3-boronato-5-nitro)benzoyl-Nε-(4-dimethylamino-3,5-dinitro)benzoyllysine;
N-α-(3,4-dihydroxybenzoyl)-N-ε-(5-dimethylaminonaphthalene-1-sulfonyl)-lysine;
N-α-(3,4-dihydroxybenzoyl)-N-ε-(5-dimethylaminonaphthalene-1-sulfonyl)-lysine N-3-(methacrylamido)propylcarboxamide; and
N-α-(3-boronato-5-nitro)benzoyl-N-ε-(4-dimethylamino-3,5-dinitro)benzoyllysine N-3-(methacrylamido)propyl-carboxamide.
